(19) United States
(12) Patent Application Publication
(10) Pub. No.: US 2023/0131274 A1
(43) Pub. Date: Apr. 27, 2023

(54) P-TYPE ORGANIC SEMICONDUCTOR MATERIAL, MANUFACTURING METHOD OF SAME, AND DISPLAY PANEL

(71) Applicant: Wuhan China Star Optoelectronics Semiconductor Display Technology Co., Ltd., Wuhan (CN)

(72) Inventor: Yanjie WANG, Wuhan (CN)

(21) Appl. No.: 17/047,471

(22) PCT Filed: Jun. 15, 2020
(86) PCT No.: PCT/CN2020/096202

§ 371 Date: Oct. 14, 2020

(30) Foreign Application Priority Data
May 27, 2020 (CN) 202010460836.5

(51) Int. Cl. H10K 85/60; C07D 487/22; C07D 487/14; C07D 487/04

(52) U.S. Cl. CPC H10K 85/6572; C07D 487/22; C07D 487/14; C07D 487/04; H10K 50/17

(57) ABSTRACT

A p-type organic semiconductor material, a manufacturing method of the same, and a display panel are provided by the present application. Substitutes are configured to replace hydrogens on ring in a molecular structure of the p-type organic semiconductor material of the present application, therefore a LUMO energy level of the p-type organic semiconductor material obtained is reduced.

### BACKGROUND OF INVENTION

### Field of Invention

The present application relates to a field of organic light emitting diodes, and particularly relates to a p-type organic semiconductor material, a manufacturing method of same, and a display panel.

### Description of Prior Art

A known kind of organic light emitting diode includes an anode, a hole injection layer, a hole transport layer, a light-emitting layer, an electron injection layer, an electron transport layer, and a cathode. When the organic light emitting diodes are charged with electricity, electron carriers are generated from the cathode and injected into the electron injection layer, and reach the light-emitting layer after passing through the electron transport layer; hole carriers are generated from the anode and injected into the hole injection layer, and reach the light-emitting layer after passing through the hole transport layer; the electron carriers and the hole carriers combine in the light-emitting layer or an interface between the light-emitting layer and the transport layers to generate exciton and emit light. As highest occupied molecular orbital (HOMO) of hole injection layer materials and HOMO of anode materials, such as indium tin oxide (ITO) differ substantially, a relatively high barrier needs to be crossed for holes reaching the hole injection layer from ITO, resulting in an increase in driving voltage of devices and power consumption, and a reduction of device lifespan. To solve this problem, a known method is to dope a p-type material into the hole injection layer to produce a p-type doped material so as to lower the barrier between the ITO and the hole injection layer to improve an injection efficiency of the holes. However, such p-type materials are hard to synthesize and have high cost.

### SUMMARY OF INVENTION

In view of this, the present application provides a p-type organic semiconductor material, a manufacturing method of the same and a display panel using the p-type organic semiconductor material which can lower manufacturing costs.

A p-type organic semiconductor material is provided,

having a molecular structure represented by

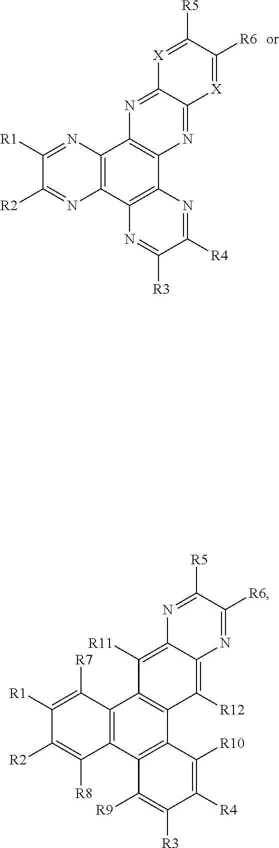

wherein X is a carbon atom or a nitrogen atom, and each of R7, R8, R8, R10, R11, R12 is selected from a first group of substituents;

when R1 and R2 are not bonded to form a ring, each of R1 and R2 is selected from the first group of substituents;

when R1 and R2 are bonded to form the ring, a structure of the ring bonded with R1 and R2 is selected from one of

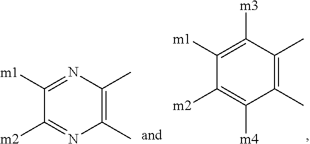

and each of m1, m2, m3, and m4 is selected from a second group of substituents;

when R3 and R4 are not bonded to form a ring, R3 and R4 are selected from the first group of substituents;

when R3 and R4 are bonded to form the ring, a structure of the ring bonded with R3 and R4 is selected from one of

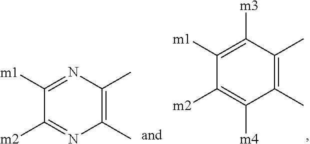

and each of m1, m2, m3, and m4 is selected from the second group of substituents;

when R5 and R6 are not bonded to form a ring, each of R5 and R6 is selected from the first group of substituents;

when R5 and R6 are bonded to form the ring, a structure of the ring bonded with R5 and R6 is selected from one of

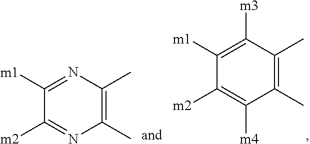

and each of m1, m2, m3, and m4 is selected from the second group of substituents;

wherein the first group of substituents consists of nitro group, nitrile group, cyano group, halogen group, halogen alkyl group, ester group, silyl group, acyl group, sulfonic group, formyl group, carbonyl group, carboxyl group, sulfoxide group, hydroxyl group, alkoxy group, amino group, aromatic amino group, acylamino group, substituted alkene, substituted phenyl group, substituted heterocyclic group; and

the second group of substituents consists of halogen, CN,

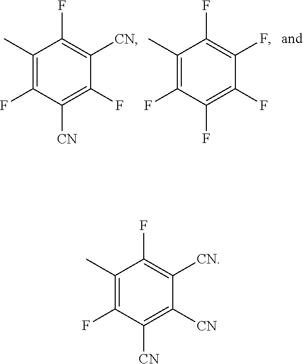

A manufacturing method of a p-type organic semiconductor material is also provided, comprising following steps:

cyclizing a first reactant containing diketone with a second reactant containing diamine to produce the p-type organic semiconductor material, wherein the p-type organic semiconductor material has a molecular structure represented by

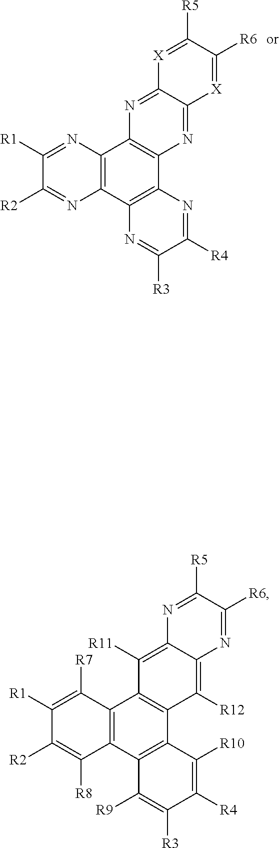

wherein X is a carbon atom or a nitrogen atom, and each of R7, R8, R8, R10, R11, R12 is selected from a first group of substituents;

when R1 and R2 are not bonded to form a ring, each of R1 and R2 is selected from the first group of substituents;

when R1 and R2 are bonded to form the ring, a structure of the ring bonded with R1 and R2 is selected from one of

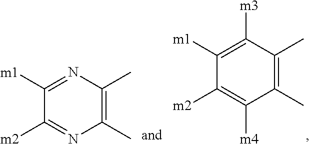

and each of m1, m2, m3, and m4 is selected from a second group of substituents;

when R3 and R4 are not bonded to form a ring, R3 and R4 are selected from the first group of substituents;

when R3 and R4 are bonded to form the ring, a structure of the ring bonded with R3 and R4 is selected from one of

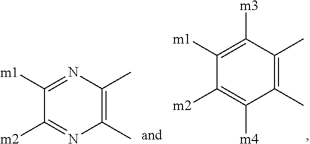

and each of m1, m2, m3, and m4 is selected from the second group of substituents;

when R5 and R6 are not bonded to form the ring, each of R5 and R6 is selected from the first group of substituents;

when R5 and R6 are c bonded to form the ring, a structure of the ring bonded with R5 and R6 is selected from one of

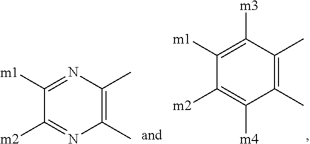

and each of m1, m2, m3, and m4 is selected from the second group of substituents;

wherein the first group of substituents consists of nitro group, nitrile group, cyano group, halogen group, halogen alkyl group, ester group, silyl group, acyl group, sulfonic group, formyl group, carbonyl group, carboxyl group, sulfoxide group, hydroxyl group, alkoxy group, amino group, aromatic amino group, acylamino group, substituted alkene, substituted phenyl group, substituted heterocyclic group; and

the second group of substituents consists of halogen, CN

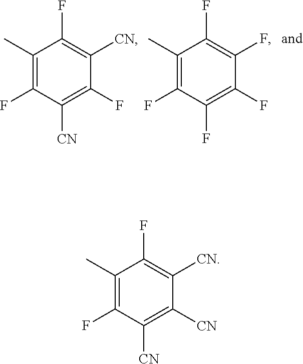

An organic light-emitting diode display panel is provided by the present application, comprising a substrate and a plurality of organic light-emitting diode (OLED) devices disposed on the substrate, wherein each of the OLED devices comprises an anode, a cathode, and a hole injection layer, a hole transport layer, a light-emitting layer, an electron transport layer and an electron injection layer sequentially stacked between the anode and the cathode, and the hole injection layer comprises the p-type organic semiconductor material of anyone of claims 1 to 4.

Substituents are configured to replace hydrogens on the rings in the molecular structure of the p-type organic semiconductor material of the present application, lowering a lowest unoccupied molecular orbital (LUMO) energy level of the p-type organic semiconductor material obtained. When the p-type organic semiconductor material of the present application is applied as a hole injection layer and a hole transport layer of the light-emitting devices, ability of the hole transport layer to form holes is enhanced, hole injection from the anode to the hole transport layer is improved, hole mobility is improved accordingly, lowering a driving voltage of the light-emitting devices. On the other hand, the p-type organic semiconductor material has a planar molecular structure, which further improves hole mobility and benefits hole injection and transport. The manufacturing method of the p-type organic semiconductor material provided by the present application requires simple synthesis steps and lower costs.

### BRIEF DESCRIPTION OF DRAWINGS

In order to more clearly illustrate the technical solution of the present application, a brief description of the drawings that are necessary for the illustration of the embodiments will be given as follows. Obviously, the drawings described below show only some embodiments of the present invention, and a person having ordinary skill in the art may also obtain other drawings based on the drawings described without making any creative effort.

FIG. 1(a) and FIG. 1(b) are simulated diagrams of 3D molecular structures of HATCN of HOMO energy level and LUMO level.

FIG. 2(a) and FIG. 2(b) are simulated diagrams of 3D molecular structures of a target product 1 of HOMO energy level and LUMO level.

FIG. 3 is a sectional view of an OLED display panel according to one embodiment of the present application.

FIG. 4 is a sectional view of the OLED device in FIG. 3.

### DETAILED DESCRIPTION OF PREFERRED EMBODIMENTS

The present disclosure is further described in detail below with reference to the accompanying drawings and embodiments. Obviously, the following described embodiments are only part of the present disclosure but not all. A person having ordinary skill in the art may obtain other embodiments based on the embodiments provided in the present disclosure without making any creative effort, which all belong to the scope of the present disclosure.

The p-type organic semiconductor material of the present application can be applied in a hole injection layer and a hole transport layer of an OLED device 30.

The p-type organic semiconductor material of the present application has a molecular structure represented by

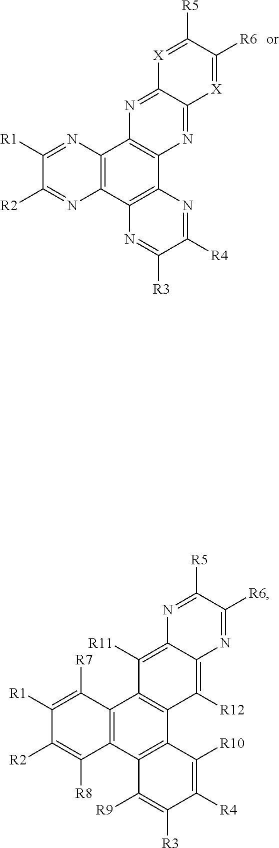

wherein X is a carbon atom or a nitrogen atom; each of R7, R8, R8, R10, R11, R12 is selected from a first group of substituents;

when R1 and R2 are not bonded to form the ring, each of R1 and R2 is selected from the first group of substituents;

when R1 and R2 are bonded to form the ring, a structure of the ring bonded with R1 and R2 is selected from one of

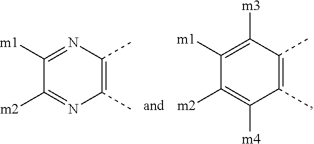

and each of m1, m2, m3 and m4 is selected from the second group of substituents;

when R3 and R4 are not bonded to form a ring, R3 and R4 are selected from the first group of substituents;

when R3 and R4 are bonded to form the ring, a structure of the ring bonded with R3 and R4 is selected from one of

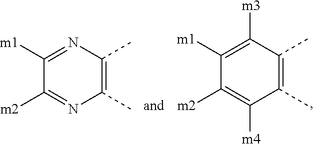

and each of m1, m2, m3, and m4 is selected from the second group of substituents;

when R5 and R6 are not bonded to form a ring, each of R5 and R6 is selected from the first group of substituents;

when R5 and R6 are bonded to form the ring, a structure of the ring bonded with R5 and R6 is selected from one of

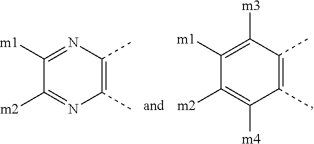

and each of m1, m2, m3, and m4 is selected from the second group of substituents;

wherein the first group of substituents consists of nitro group, nitrile group, cyano group, halogen group, halogen alkyl group, ester group, silyl group, acyl group, sulfonic group, formyl group, carbonyl group, carboxyl group, sulfoxide group, hydroxyl group, alkoxy group, amino group, aromatic amino group, acylamino group, substituted alkene, substituted phenyl group, substituted heterocyclic group; and

the second group of substituents consists of halogen, CN,

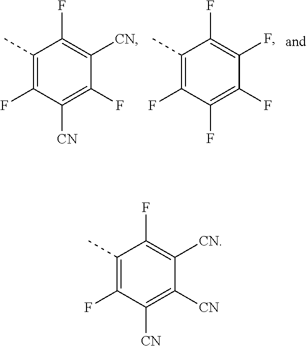

In one embodiment, the p-type organic semiconductor material is one of following compounds:

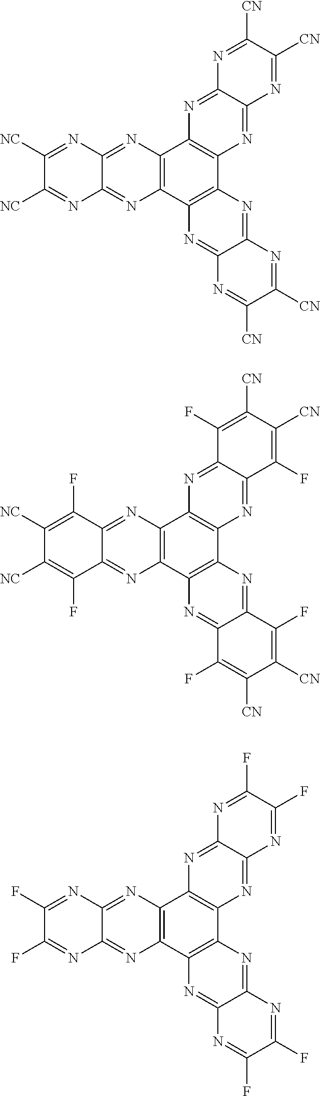

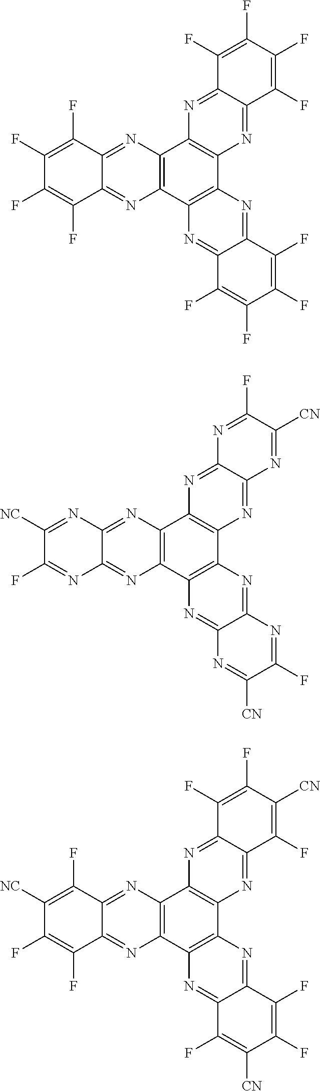

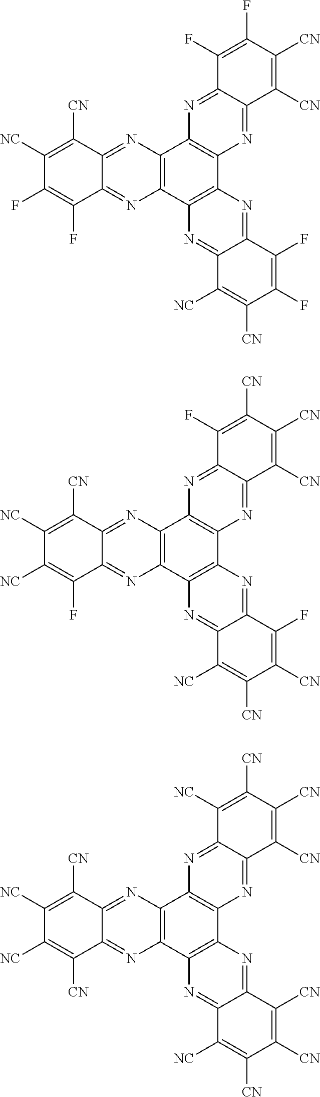

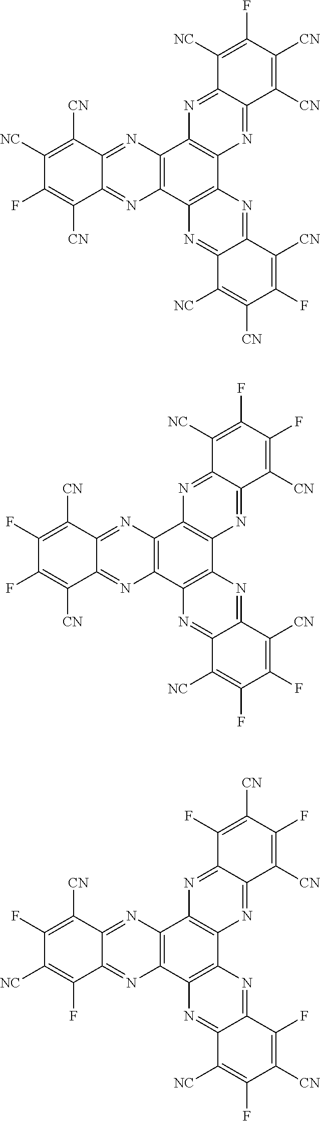 [SMILES: N#Cc1c(F)c(C#N)c2nc3c4nc5c(C#N)c(C#N)c(F)c(C#N)c5nc4c4nc5c(C#N)c(C#N)c(F)c(C#N)c5nc4c3nc2c1C#N ; N#Cc1c(F)c(F)c(C#N)c2nc3c(nc12)c1nc2c(C#N)c(F)c(F)c(C#N)c2nc1c1nc2c(C#N)c(F)c(F)c(C#N)c2nc31 ; N#Cc1c(F)c(C#N)c2nc3c4nc5c(F)c(C#N)c(F)c(C#N)c5nc4c4nc5c(F)c(C#N)c(F)c(C#N)c5nc4c3nc2c1F]

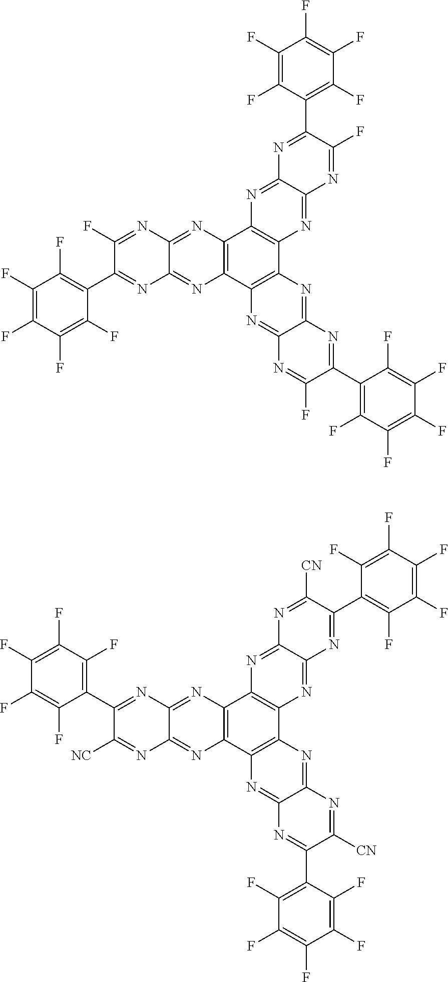 [SMILES: Fc1nc2nc3c4nc5nc(-c6c(F)c(F)c(F)c(F)c6F)c(F)nc5nc4c4nc5nc(-c6c(F)c(F)c(F)c(F)c6F)c(F)nc5nc4c3nc2nc1-c1c(F)c(F)c(F)c(F)c1F ; N#Cc1nc2nc3c4nc5nc(-c6c(F)c(F)c(F)c(F)c6F)c(C#N)nc5nc4c4nc5nc(-c6c(F)c(F)c(F)c(F)c6F)c(C#N)nc5nc4c3nc2nc1-c1c(F)c(F)c(F)c(F)c1F]

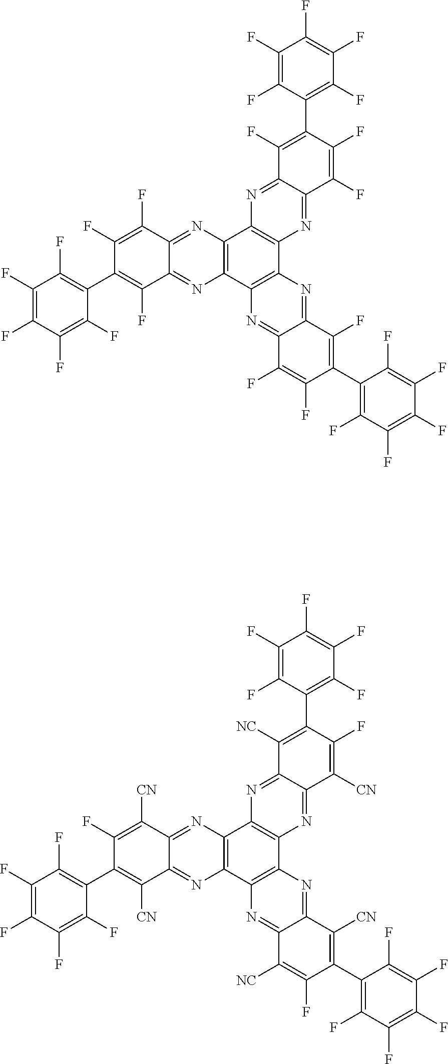 [SMILES: Fc1c(F)c(F)c(-c2c(F)c(F)c3nc4c5nc6c(F)c(-c7c(F)c(F)c(F)c(F)c7F)c(F)c(F)c6nc5c5nc6c(F)c(-c7c(F)c(F)c(F)c(F)c7F)c(F)c(F)c6nc5c4nc3c2F)c(F)c1F ; N#Cc1c(F)c(-c2c(F)c(F)c(F)c(F)c2F)c(C#N)c2nc3c4nc5c(C#N)c(F)c(-c6c(F)c(F)c(F)c(F)c6F)c(C#N)c5nc4c4nc5c(C#N)c(F)c(-c6c(F)c(F)c(F)c(F)c6F)c(C#N)c5nc4c3nc12]

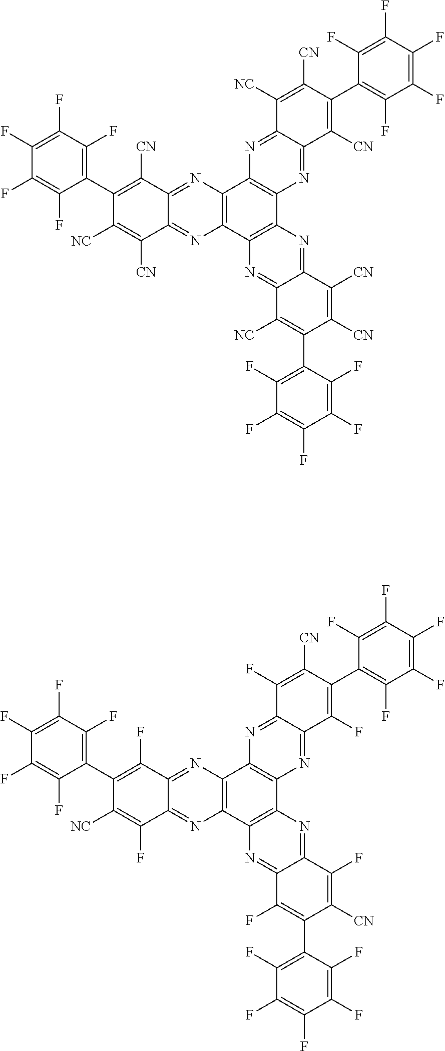 [SMILES: N#Cc1c(-c2c(F)c(F)c(F)c(F)c2F)c(C#N)c2nc3c4nc5c(C#N)c(C#N)c(-c6c(F)c(F)c(F)c(F)c6F)c(C#N)c5nc4c4nc5c(C#N)c(C#N)c(-c6c(F)c(F)c(F)c(F)c6F)c(C#N)c5nc4c3nc2c1C#N ; N#Cc1c(-c2c(F)c(F)c(F)c(F)c2F)c(F)c2nc3c4nc5c(F)c(C#N)c(-c6c(F)c(F)c(F)c(F)c6F)c(F)c5nc4c4nc5c(F)c(C#N)c(-c6c(F)c(F)c(F)c(F)c6F)c(F)c5nc4c3nc2c1F]

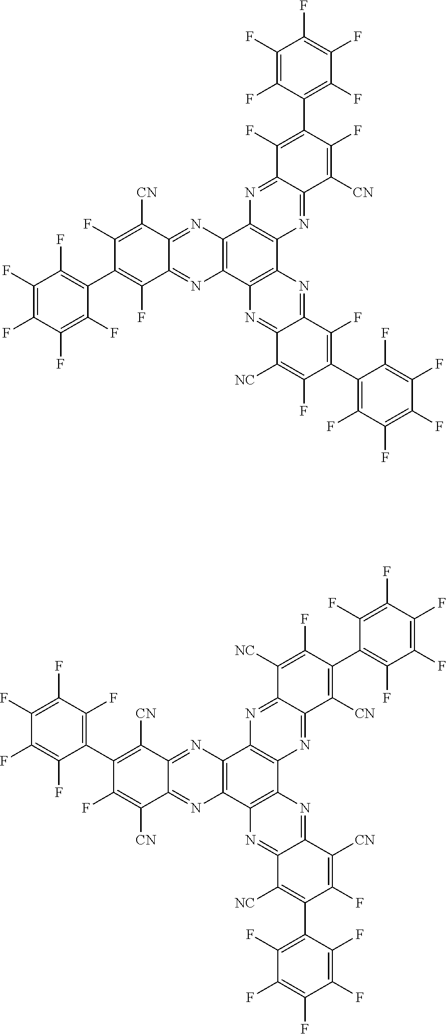 [SMILES: N#Cc1c(F)c(-c2c(F)c(F)c(F)c(F)c2F)c(F)c2nc3c4nc5c(C#N)c(F)c(-c6c(F)c(F)c(F)c(F)c6F)c(F)c5nc4c4nc5c(C#N)c(F)c(-c6c(F)c(F)c(F)c(F)c6F)c(F)c5nc4c3nc12 ; N#Cc1c(F)c(-c2c(F)c(F)c(F)c(F)c2F)c(C#N)c2nc3c4nc5c(C#N)c(F)c(-c6c(F)c(F)c(F)c(F)c6F)c(C#N)c5nc4c4nc5c(C#N)c(F)c(-c6c(F)c(F)c(F)c(F)c6F)c(C#N)c5nc4c3nc12]

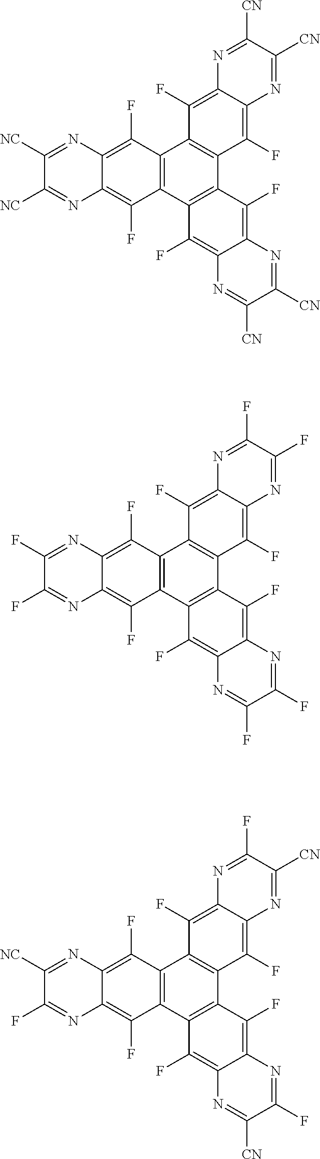 [SMILES: N#Cc1nc2c(F)c3c4c(F)c5nc(C#N)c(C#N)nc5c(F)c4c4c(F)c5nc(C#N)c(C#N)nc5c(F)c4c3c(F)c2nc1C#N ; Fc1nc2c(F)c3c4c(F)c5nc(F)c(F)nc5c(F)c4c4c(F)c5nc(F)c(F)nc5c(F)c4c3c(F)c2nc1F ; N#Cc1nc2c(F)c3c4c(F)c5nc(F)c(C#N)nc5c(F)c4c4c(F)c5nc(F)c(C#N)nc5c(F)c4c3c(F)c2nc1F]

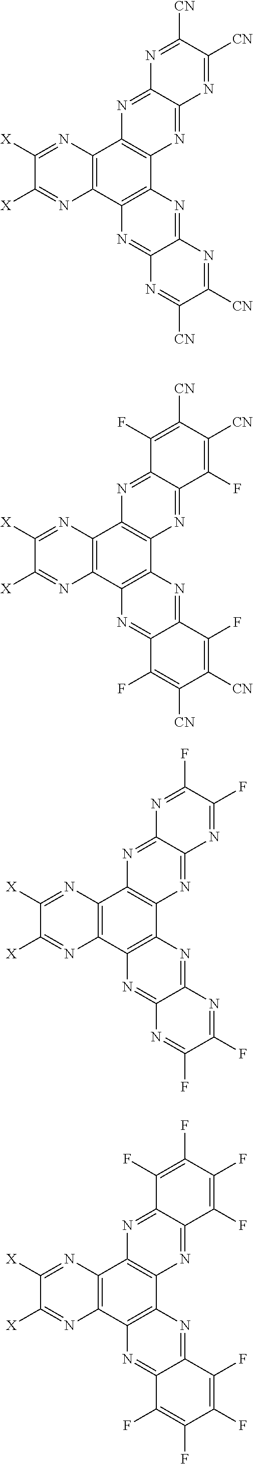

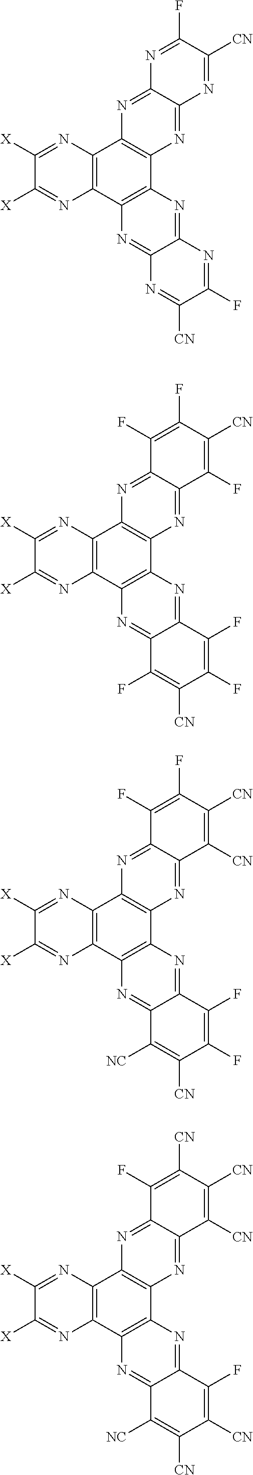

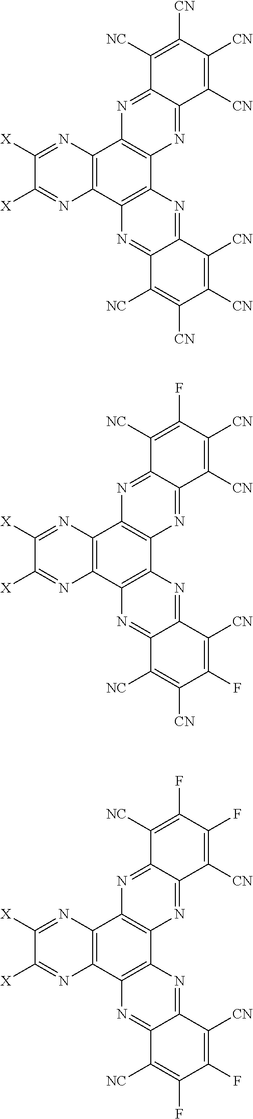

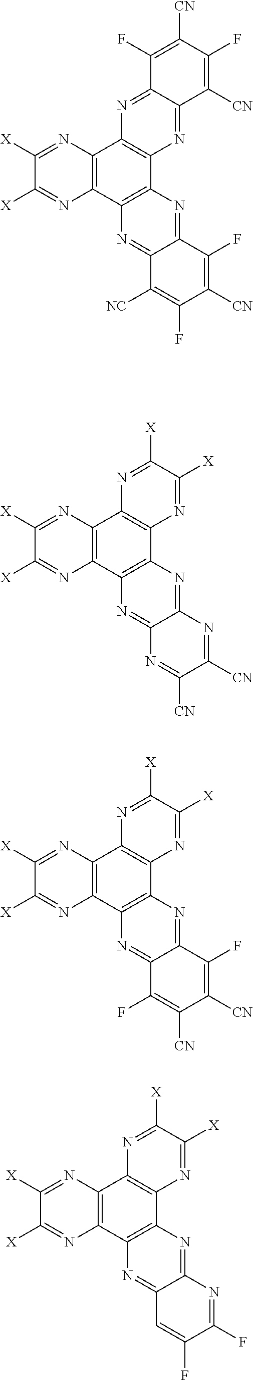

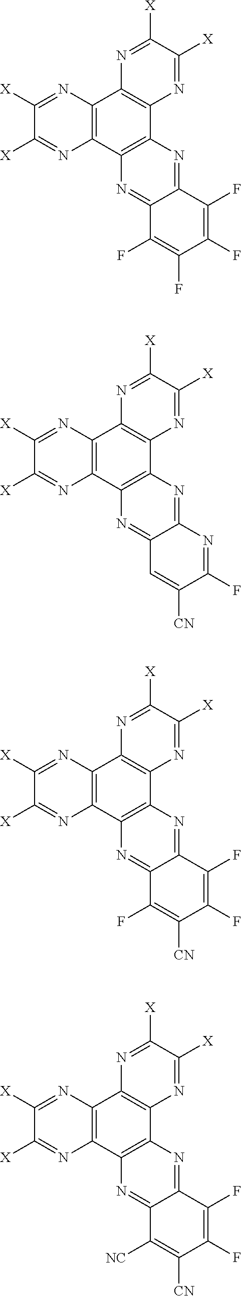

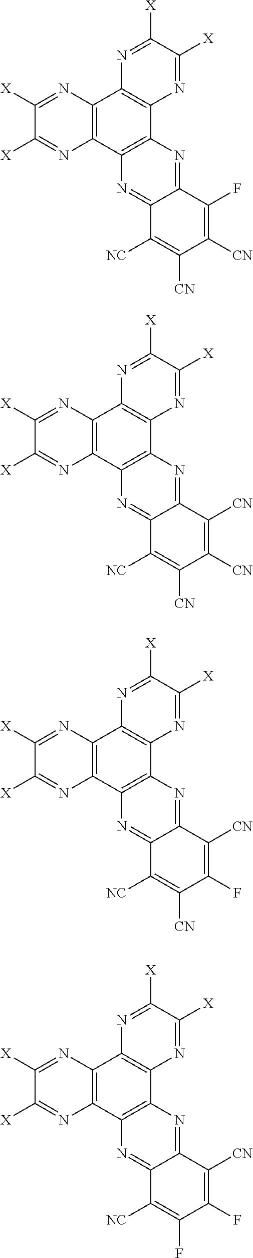

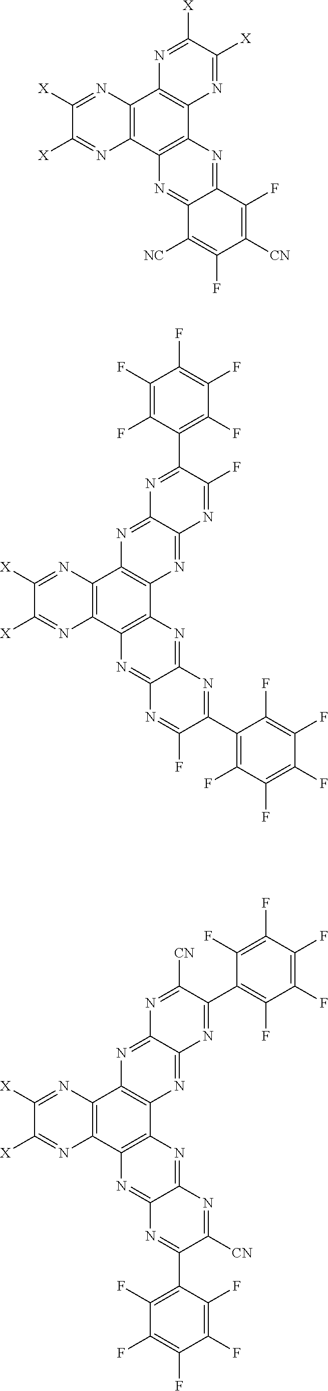

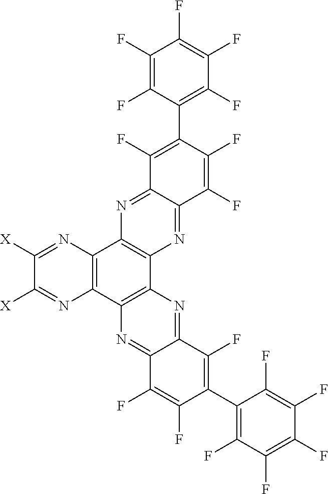

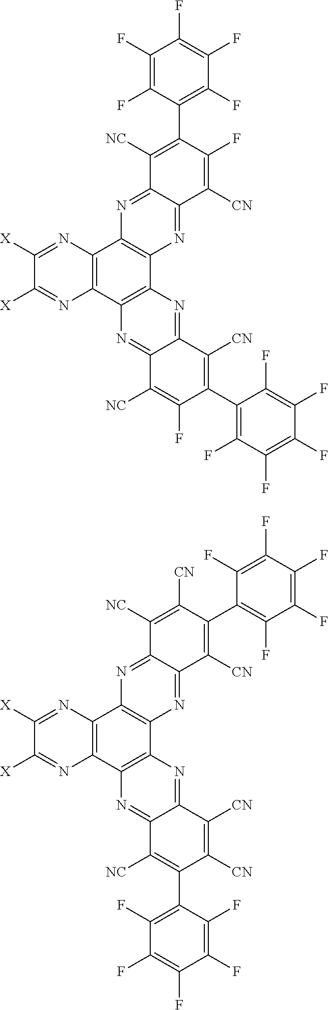

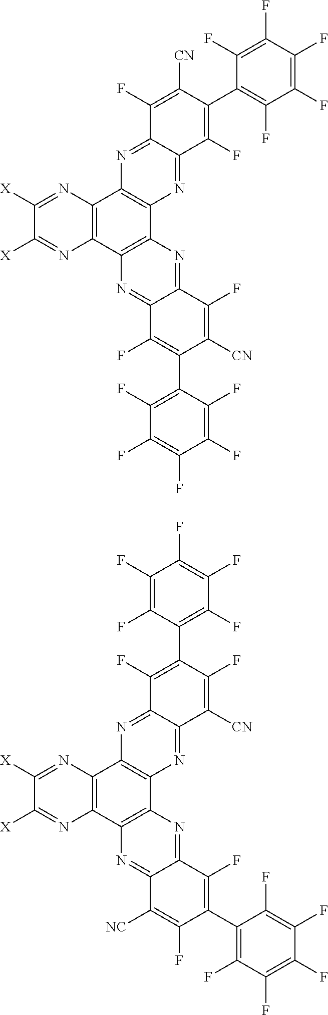

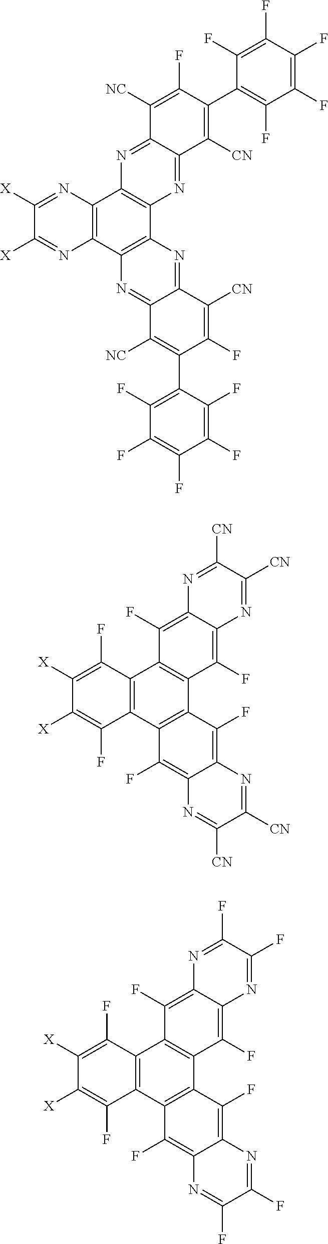

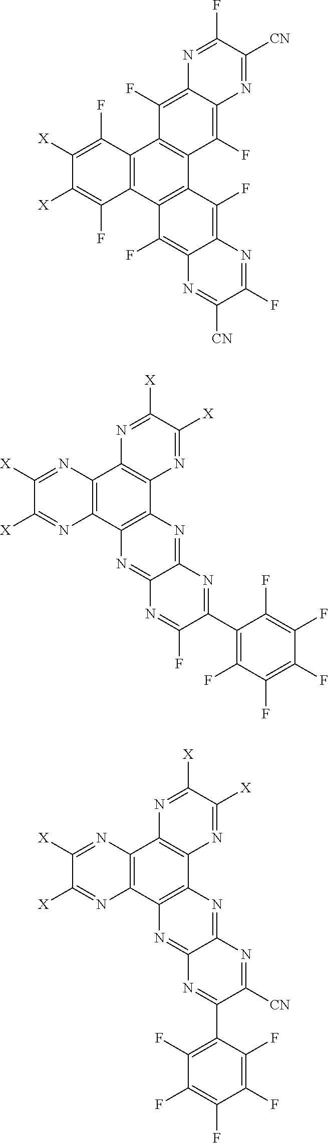

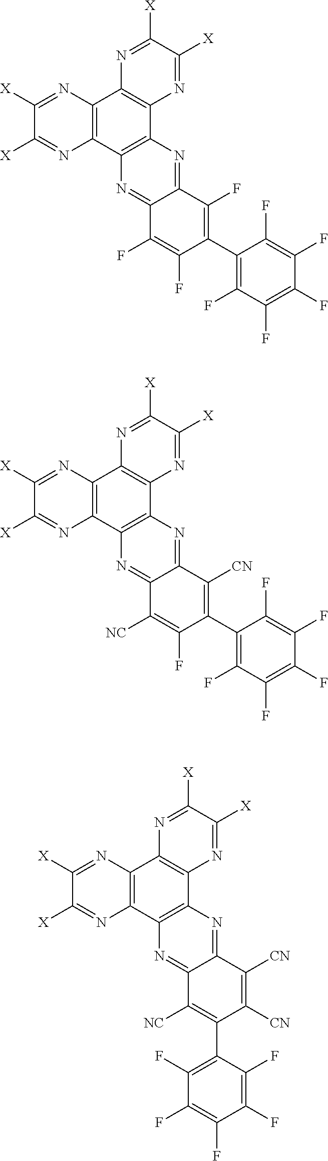

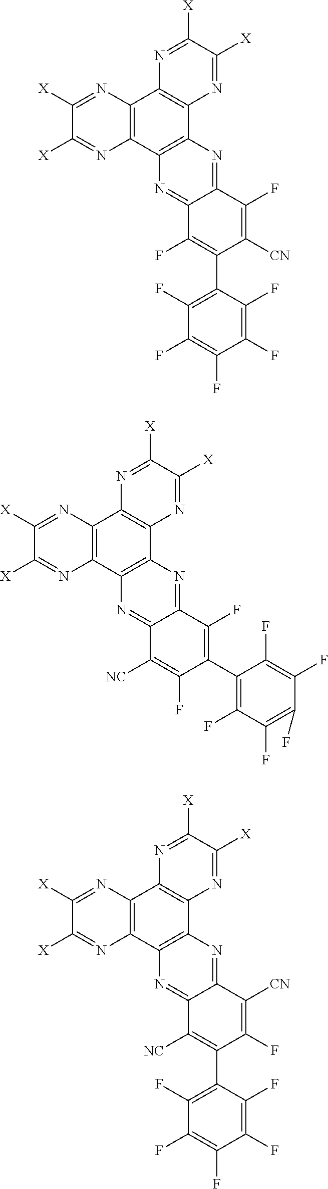

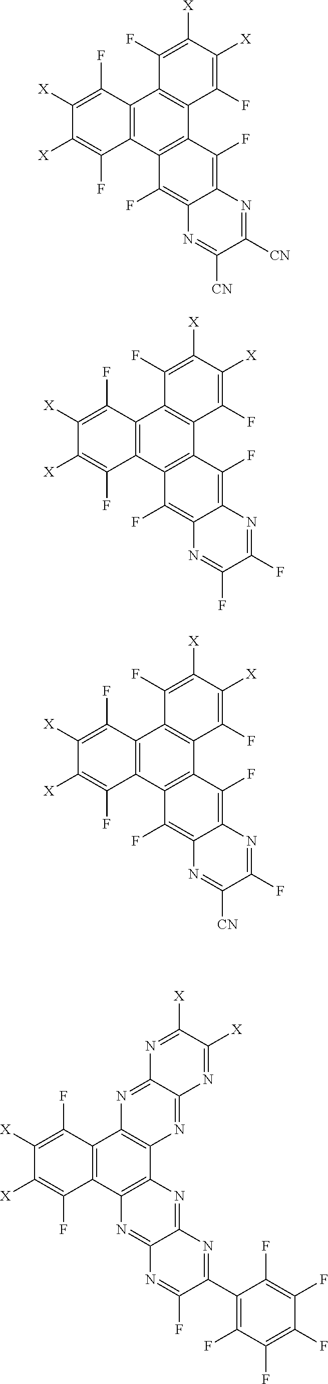

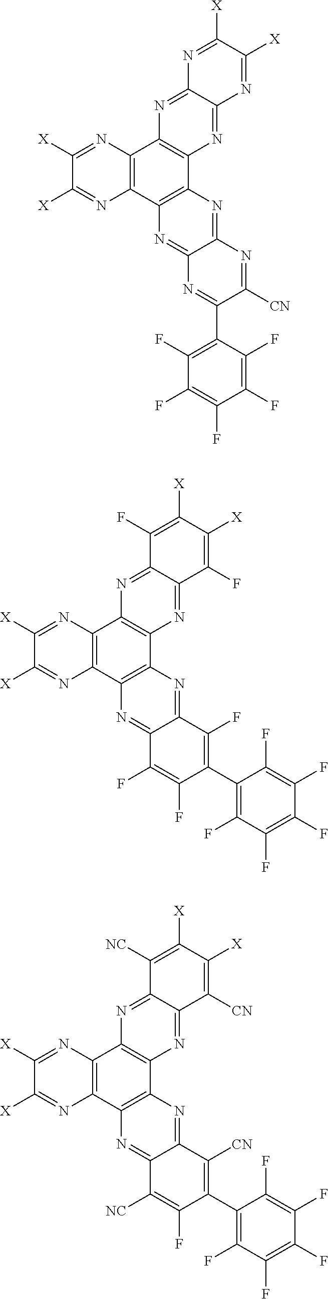

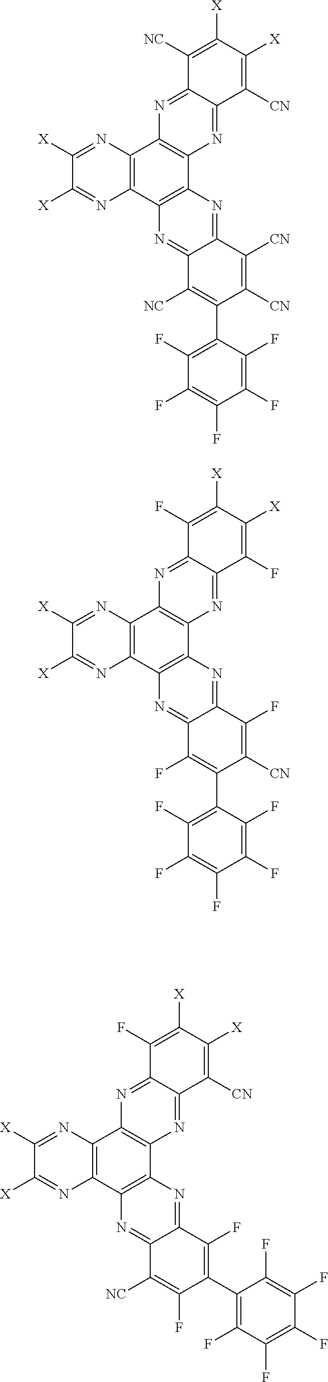

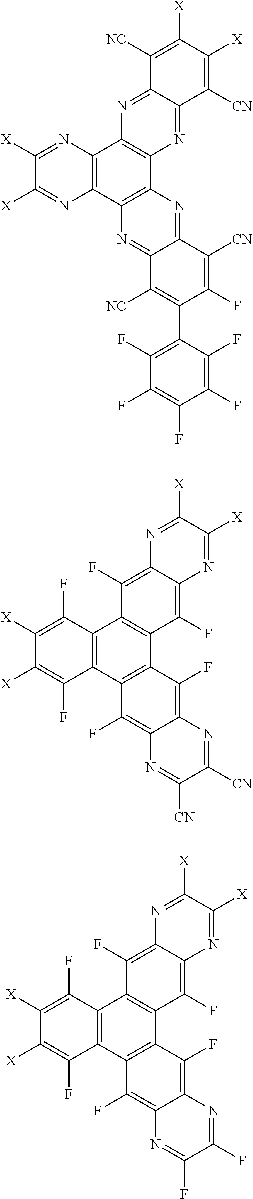

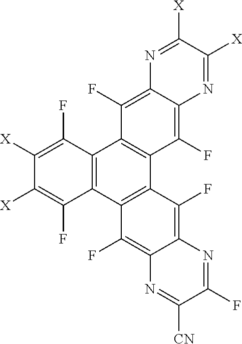

wherein group X is one or more of —CN, —F,

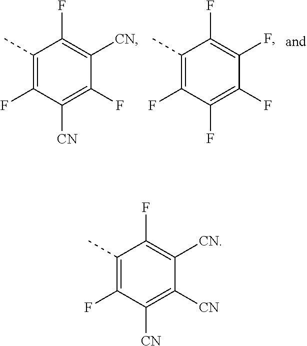

Substituents are configured to replace hydrogens on the rings in the molecular structure of the p-type organic semiconductor material of the present application, lowering a lowest unoccupied molecular orbital (LUMO) energy level of the p-type organic semiconductor material obtained. When the p-type organic semiconductor material of the present application is applied as a hole injection layer and a hole transport layer of the light-emitting devices, ability of the hole transport layer to form holes is enhanced, hole injection from the anode to the hole transport layer is improved, hole mobility is improved accordingly, lowering a driving voltage of the light-emitting devices.

On the other hand, the p-type organic semiconductor material has a planar molecular structure, which further improves hole mobility and benefits hole injection and transport.

A manufacturing method of a p-type organic semiconductor material is also provided in the present application, comprising steps of: cyclizing a first reactant containing diketone with a second reactant containing diamine to produce a p-type organic semiconductor material,

wherein the p-type organic semiconductor material has a molecular structure represented by

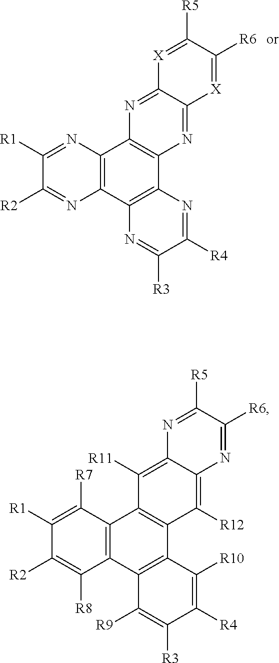

wherein X is a carbon atom or a nitrogen atom; each of R7, R8, R8, R10, R11, R12 is selected from a first group of substituents;

when R1 and R2 are not bonded to form a ring, each of R1 and R2 is selected from the first group of substituents;

when R1 and R2 are bonded to form the ring, a structure of the ring bonded with R1 and R2 is selected from one of

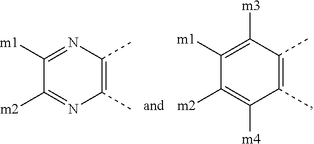

and each of m1, m2, m3, and m4 is selected from a second group of substituents;

when R3 and R4 are not bonded to form a ring, R3 and R4 are selected from the first group of substituents;

when R3 and R4 are bonded to form the ring, a structure of the ring bonded with R3 and R4 is selected from one of

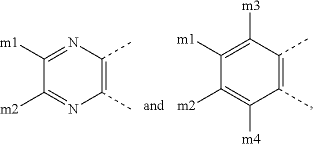

and each of m1, m2, m3, and m4 is selected from the second group of substituents;

when R5 and R6 are not bonded to form the ring, each of R5 and R6 is selected from the first group of substituents;

when R5 and R6 are c bonded to form the ring, a structure of the ring bonded with R5 and R6 is selected from one of

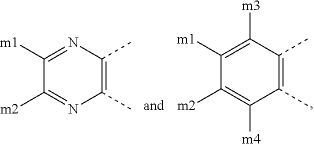

and each of m1, m2, m3, and m4 is selected from the second group of substituents;

wherein the first group of substituents consists of nitro group, nitrile group, cyano group, halogen group, halogen alkyl group, ester group, silyl group, acyl group, sulfonic group, formyl group, carbonyl group, carboxyl group, sulfoxide group, hydroxyl group, alkoxy group, amino group, aromatic amino group, acylamino group, substituted alkene, substituted phenyl group, substituted heterocyclic group; and

the second group of substituents consists of halogen, CN

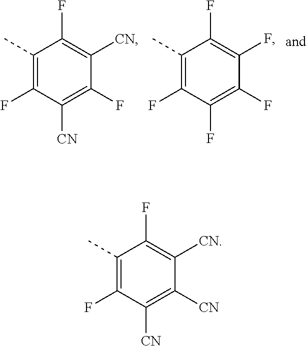

In one embodiment of this application, the first reactant has a molecular structure represented by

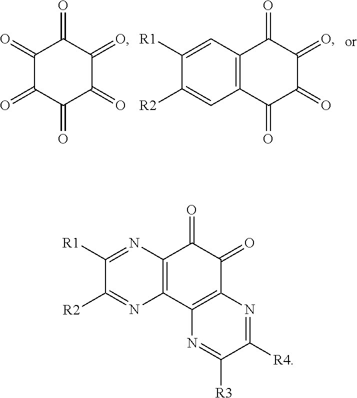

When the first reactant is

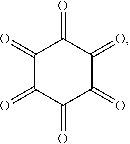 [SMILES: O=C1C(=O)C(=O)C(=O)C(=O)C1=O]

the second reactant comprises

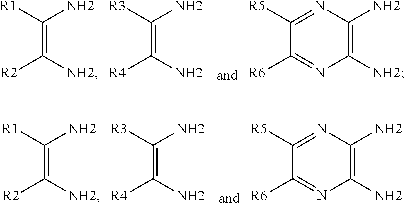

can be same or different.

When the first reactant is

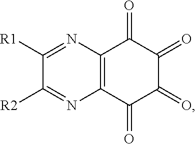

the second reactant comprises

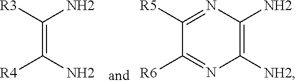

and

when the first reactant is

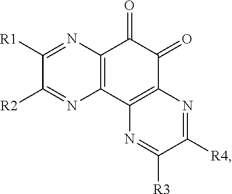

the second reactant comprises

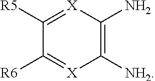

It can be noted that,

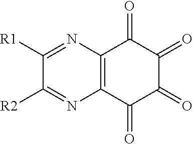

can be produced by cyclizing

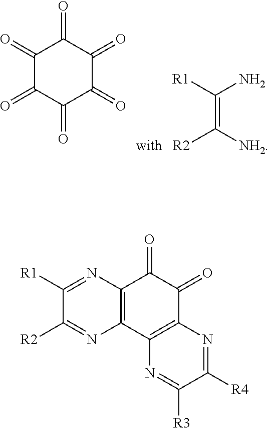

can be produced by one or two steps of cyclization reaction of

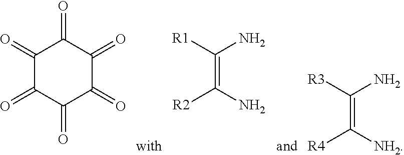

In one embodiment of this application, the first reactant has a molecular structure represented by

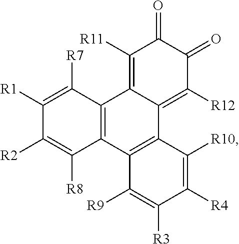

and the second reactant has a molecular structure represented by

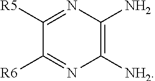

In one embodiment of this application, the manufacturing method further includes producing

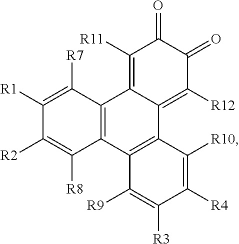

through substitution reaction of

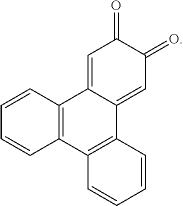 [SMILES: O=C1C=c2c(c3ccccc3c3ccccc23)=CC1=O]

In one embodiment of this application, a molar ratio of the first reactant to the second reactant is 1:1 to 1:10.

In one embodiment of this application, a reaction condition of the step of cyclizing a first reactant containing diketone with a second reactant containing diamine is as below: acid catalyst, for example acetic acid, sulphuric acid, hydrochloric acid is added under protection of nitrogen, and a reaction temperature is 25° C. to 100° C., a reaction time is 1 hour to 12 hours.

The manufacturing method of the p-type organic semiconductor material provided by the present application requires simple synthesis steps and lower costs.

The manufacturing method of the p-type organic semiconductor material of the present application will be described in detail through the embodiments as below.

### Embodiment 1

Cyclohexanone (0.98 g, 10 mmol), 5,6-diaminopiperidine-2,3-dicyano (12.01 g, 75 mmol), and a catalyst are added into a reaction vessel. Specifically, acetic acid (100 ml) can be used as the catalyst.

Reaction lasts for 2 hours by heating under the protection of argon.

Heat filtration, washing, and drying are performed on an obtained mixture. A solvent used for washing can be hot acetic acid.

Target product 1 is obtained by separation and refinement.

A method of separation and refinement can be as following: a chromatography is performed to a crude product using a silica gel column of 200 to 300 mesh, an eluent is dichloromethane (DCM), decolorizing, evaporation by rotary distillation, and vacuum drying are performed, and a dark brown solid is obtained, sublimation is performed, and a light yellow solid is obtained, which is the target product 1

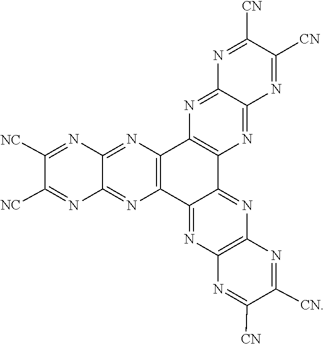 [SMILES: N#Cc1nc2nc3c4nc5nc(C#N)c(C#N)nc5nc4c4nc5nc(C#N)c(C#N)nc5nc4c3nc2nc1C#N]

Mass of the target product 1 is 2.68 g and yield is 81%.

HRMS [M+H]+ calcd. for C24N18: 540.0553; found: 540.0534.

A chemical equation of the reaction is:

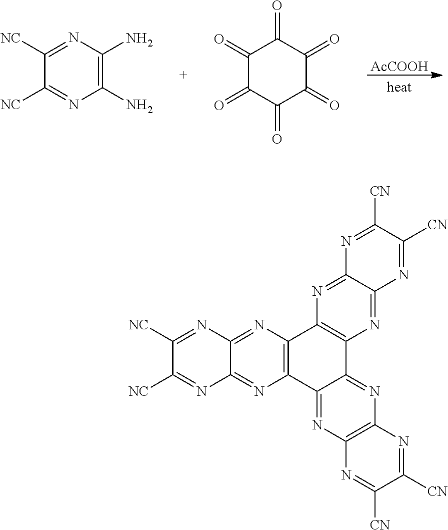 [SMILES: "AcCOOH" ; N#Cc1nc(N)c(N)nc1C#N ; O=C1C(=O)C(=O)C(=O)C(=O)C1=O ; N#Cc1nc2nc3c4nc5nc(C#N)c(C#N)nc5nc4c4nc5nc(C#N)c(C#N)nc5nc4c3nc2nc1C#N]

### Embodiment 2

(0.98 g, 10 mmol), 4,5-diamino-3,6-difluorophthalonitrile (14.55 g, 75 mmol), and acetic acid (100 ml) are added into a reaction vessel. Reaction lasts for 2 hours by heating under the protection of argon. Heat filtration, washing, and drying are performed on an obtained mixture. A solvent used for washing can be hot acetic acid. Target product 2 is obtained by separation and refinement.

A method of separation and refinement can be as following: a chromatography is performed to a crude product using a silica gel column of 200 to 300 mesh, an eluent is dichloromethane (DCM), decolorizing, evaporation by rotary distillation, and vacuum drying are performed, and a dark brown solid is obtained, sublimation is performed, and a light yellow solid is obtained, which is the target product 2

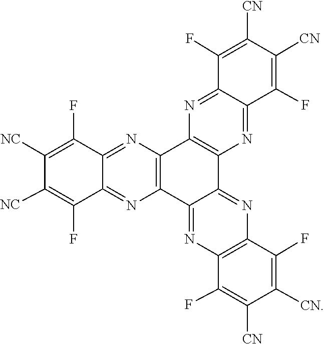 [SMILES: N#Cc1c(C#N)c(F)c2nc3c(nc2c1F)c1nc2c(F)c(C#N)c(C#N)c(F)c2nc1c1nc2c(F)c(C#N)c(C#N)c(F)c2nc31]

Mass of the target product 2 is 2.63 g and yield is 82%.

HRMS [M+H]+ calcd. for C30F6N12: 642.0273; found: 642.0277.

A chemical equation of the reaction is as follows:

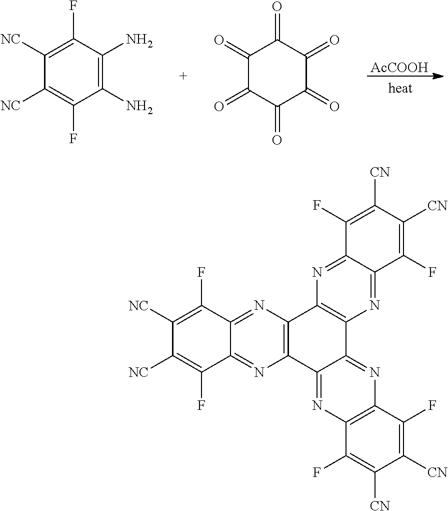 [SMILES: "AcCOOH" ; N#Cc1c(F)c(N)c(N)c(F)c1C#N ; O=C1C(=O)C(=O)C(=O)C(=O)C1=O ; N#Cc1c(C#N)c(F)c2nc3c(nc2c1F)c1nc2c(F)c(C#N)c(C#N)c(F)c2nc1c1nc2c(F)c(C#N)c(C#N)c(F)c2nc31]

### Embodiment 3

A manufacturing method of a p-type organic semiconductor material of the embodiment 3 includes steps of:

(1) Making compound 1

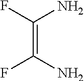

react with compound 2

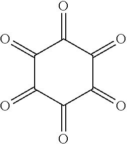 [SMILES: O=C1C(=O)C(=O)C(=O)C(=O)C1=O]

to produce a mediate product

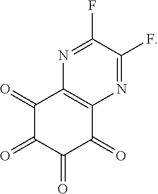 [SMILES: O=c1c(=O)c(=O)c2nc(F)c(F)nc2c1=O]

(2) Making the mediate product

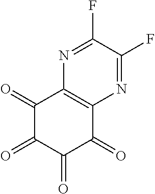 [SMILES: O=c1c(=O)c(=O)c2nc(F)c(F)nc2c1=O]

react with compound

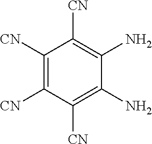

to produce a target product 3

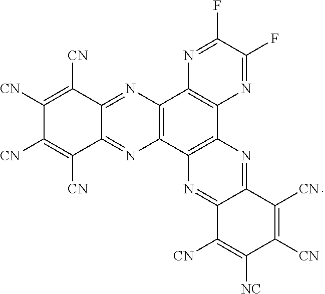

A chemical equation of the reaction is as follows:

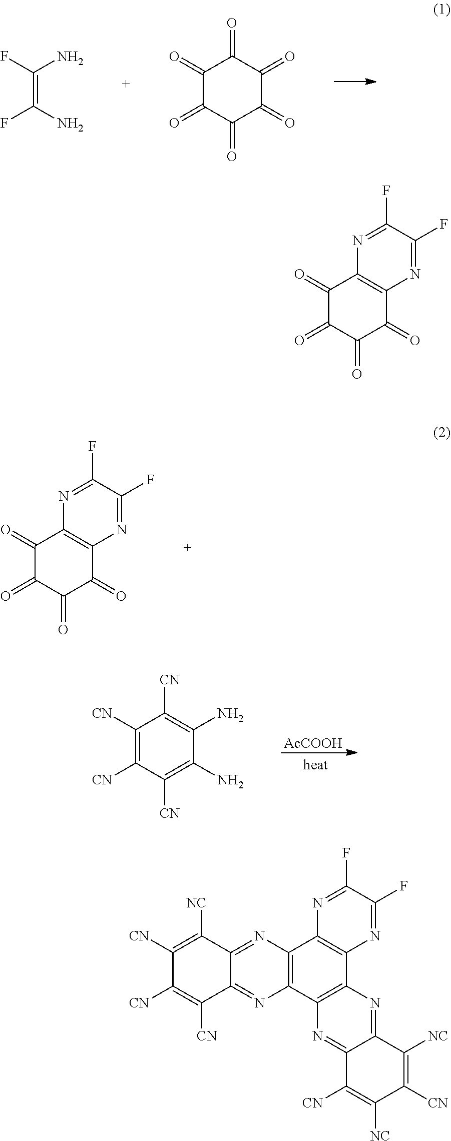

The same reaction conditions and purification methods of the embodiment 1 and 2 can be used in the embodiment 3. The detailed descriptions are omitted herein.

In another embodiment, making compound

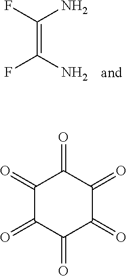 [SMILES: N/C(F)=C(\N)F ; O=C1C(=O)C(=O)C(=O)C(=O)C1=O]

react with compound

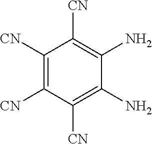

to produce the target product 3 by one step cyclizing reaction, and then purified by refinement.

### Embodiment 4

A manufacturing method of a p-type organic semiconductor material of the embodiment 4 includes steps of:

(1) Making compound 1

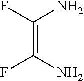

react with compound 2

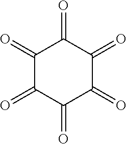 [SMILES: O=C1C(=O)C(=O)C(=O)C(=O)C1=O]

to produce a first mediate product

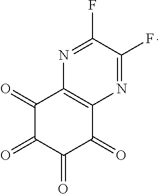 [SMILES: O=c1c(=O)c(=O)c2nc(F)c(F)nc2c1=O]

(2) Making the first mediate product

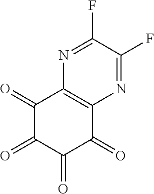 [SMILES: O=c1c(=O)c(=O)c2nc(F)c(F)nc2c1=O]

react with compound

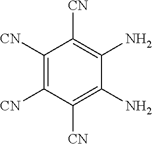

to produce a second mediate product

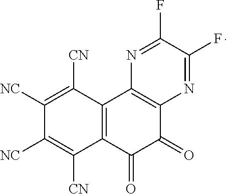 [SMILES: N#Cc1c(C#N)c(C#N)c2c(c1C#N)C(=O)C(=O)c1nc(F)c(F)nc1-2]

(3) Making the second mediate product

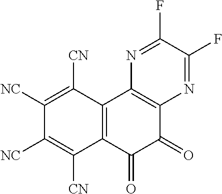 [SMILES: N#Cc1c(C#N)c(C#N)c2c(c1C#N)C(=O)C(=O)c1nc(F)c(F)nc1-2]

react with compound

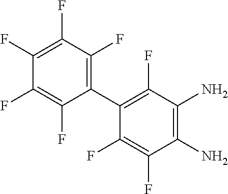 [SMILES: Nc1c(N)c(F)c(-c2c(F)c(F)c(F)c(F)c2F)c(F)c1F]

to produce a target product 4

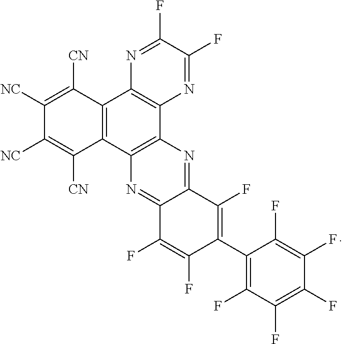 [SMILES: N#Cc1c(C#N)c(C#N)c2c3nc4c(F)c(F)c(-c5c(F)c(F)c(F)c(F)c5F)c(F)c4nc3c3nc(F)c(F)nc3c2c1C#N]

A chemical equation of the reaction is as follows:

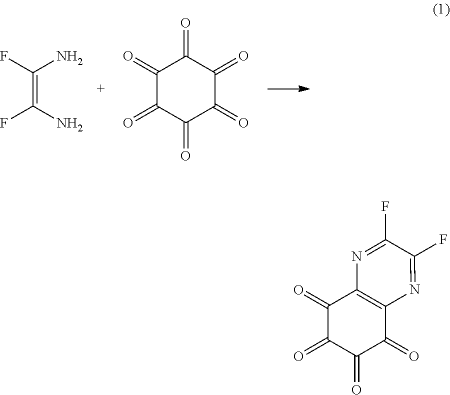 [SMILES: N/C(F)=C(\N)F ; O=C1C(=O)C(=O)C(=O)C(=O)C1=O ; O=c1c(=O)c(=O)c2nc(F)c(F)nc2c1=O]

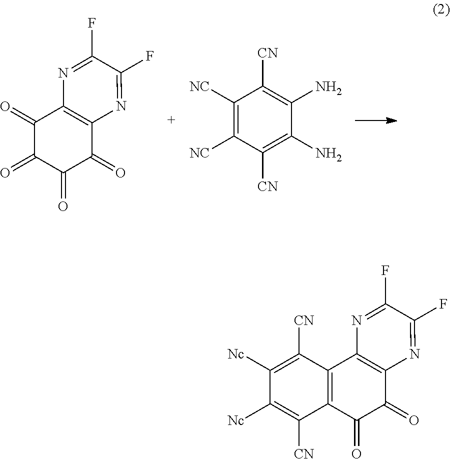

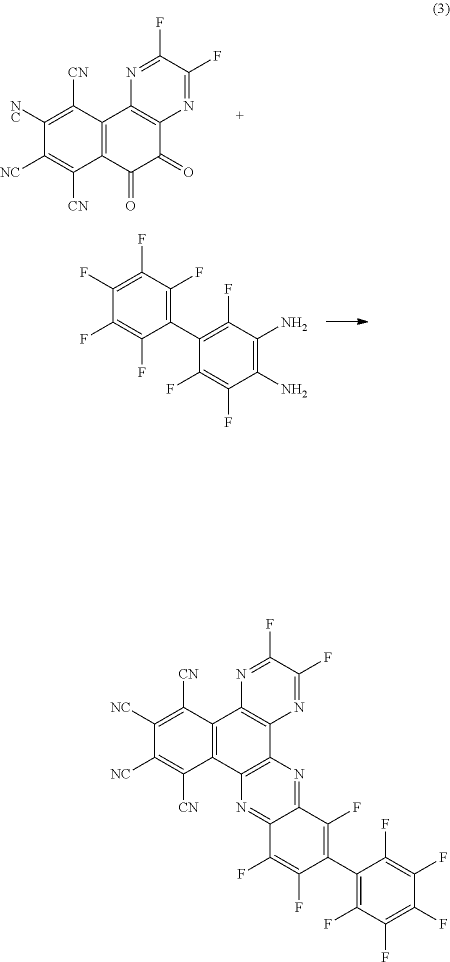

The same reaction conditions and purification methods of the embodiment 1 and 2 can be used in the embodiment 4. The detailed descriptions are omitted herein.

In another embodiment, make compounds

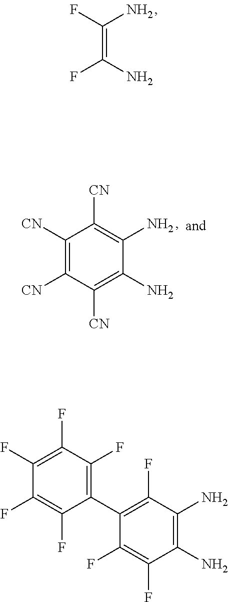

react with compound

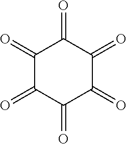 [SMILES: O=C1C(=O)C(=O)C(=O)C(=O)C1=O]

to produce the target product 4 by one step cyclizing reaction, and then purified by refinement.

### Embodiment 5

A manufacturing method of a p-type organic semiconductor material of the embodiment 5 includes steps of:

(1) Making a fluorination reaction happen between F₂ and compound

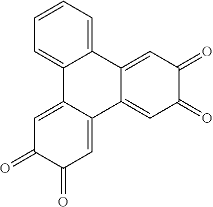 [SMILES: O=c1cc2c3ccccc3c3cc(=O)c(=O)cc3c2cc1=O]

to produce a first mediate product

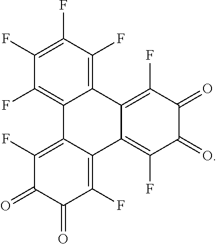 [SMILES: O=c1c(F)c2c3c(F)c(F)c(F)c(F)c3c3c(F)c(=O)c(=O)c(F)c3c2c(F)c1=O]

(2) Making the first mediate product

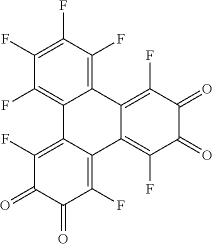 [SMILES: O=c1c(F)c2c3c(F)c(F)c(F)c(F)c3c3c(F)c(=O)c(=O)c(F)c3c2c(F)c1=O]

react with compound

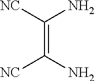

to produce a target product 5

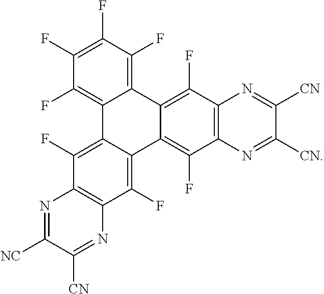 [SMILES: N#Cc1nc2c(F)c3c4c(F)c(F)c(F)c(F)c4c4c(F)c5nc(C#N)c(C#N)nc5c(F)c4c3c(F)c2nc1C#N]

The chemical equation of the reaction is as follows:

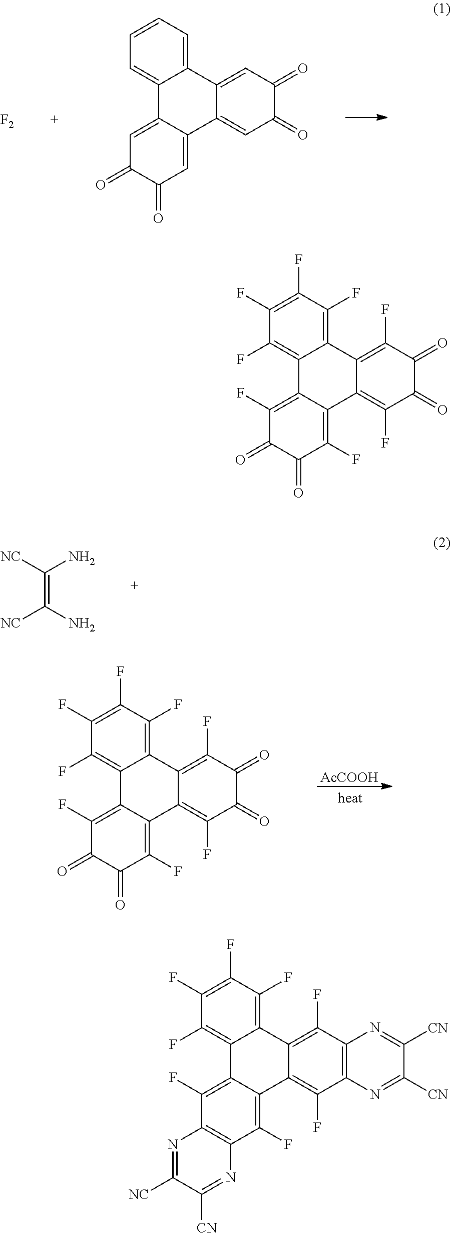

The same reaction conditions and purification methods of the embodiment 1 and 2 can be used in the embodiment 5. The detailed descriptions are omitted herein.

[Thermal Performance Tests]

A glass transition temperature (Tg) is measured by differential scanning calorimetry (DSC) and a thermal decomposition temperature (TD) at 5% weight loss is measured by thermogravimetric analysis (TGA) to each of HATCN (Hexaazatriphenylenehexacabonitrile) which is generally used in a hole injection layer, the target product 1, and the target product 2.

Results are illustrated in table 1.

TABLE 1

| | Tg (° C.) | Td (° C.) |
|---|---|---|
| HATCN | 151 | 470 |
| Target Product 1 | 166 | 545 |
| Target Product 2 | 162 | 534 |

It can be understood from table 1 that the glass transition temperature and the thermal decomposition temperature of the target product 1 and the target product 2 is higher than that of the HATCN, which means great high temperature resistance and thermal stability, and an extended lifespan in long-term use.

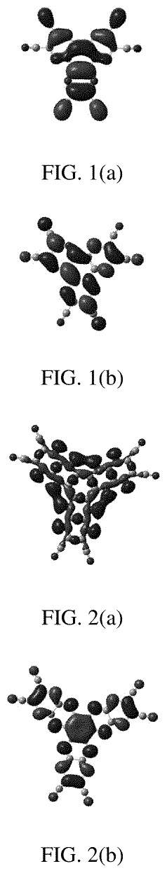

Please refer to FIG. 1(a), FIG. 1(b), FIG. 2(a), and FIG. 2(b).

FIG. 1(a), FIG. 1(b), FIG. 2(a), and FIG. 2(b) are simulated diagrams of 3D molecular structures of HATCN and the target product 1 of HOMO energy level and LUMO energy level.

The electron cloud of HOMO energy level is concentrated at electron donating groups, while the electron cloud of LUMO energy level is concentrated at electron withdrawing groups.

It can be understood from the figures that it is easier for the target product 1 of LUMO energy level than HATCN to withdraw electrons and produce holes.

In addition, theoretical calculations of photophysical data are performed on HATCN, the target product 1, and the target product 2.

Highest occupied molecular orbitals (HOMO) and lowest unoccupied molecular orbitals (LUMO) and hole mobility (μh) based on theoretical calculations are shown in Table 2 as below.

TABLE 2

| | HOMO (eV) | LUMO (eV) | μₕ (cm²/(V · s) |
|---|---|---|---|
| HATCN | −8.82 | −4.60 | 1.6*10⁻³ |
| Target Product 1 | −8.41 | −5.17 | 1.7*10⁻² |
| Target Product 2 | −8.63 | −5.02 | 1.5*10⁻² |

It can be understood from the table 2 that the hole mobility of the target product 1 and the target product 2 is much higher than HATCN, which makes the target product 1 and the target product 2 excellent materials for hole injection and hole transport.

The LUMO energy levels of p-type organic semiconductors are decreased due to substitutions of hydrogen in rings of molecular structures in the target product 1 and the target product 2 with fluorine and cyano groups. When the p-type organic semiconductor material of the present application is applied as a hole injection layer and a hole transport layer of the light-emitting devices, ability of the hole transport layer to form holes is enhanced, hole injection from the anode to the hole transport layer is improved, hole mobility is improved accordingly, lowering a driving voltage of the light-emitting devices. On the other hand, the p-type organic semiconductor material has a planar molecular structure, which further improves hole mobility and benefits hole injection and transport. In addition, the p-type organic semiconductor material requires simple synthesis steps and lower costs. Organic light emitting diodes (OLEDs) with low voltage, high efficiency, and long lifespan can be obtained if manufactured with p-type organic semiconductor materials.

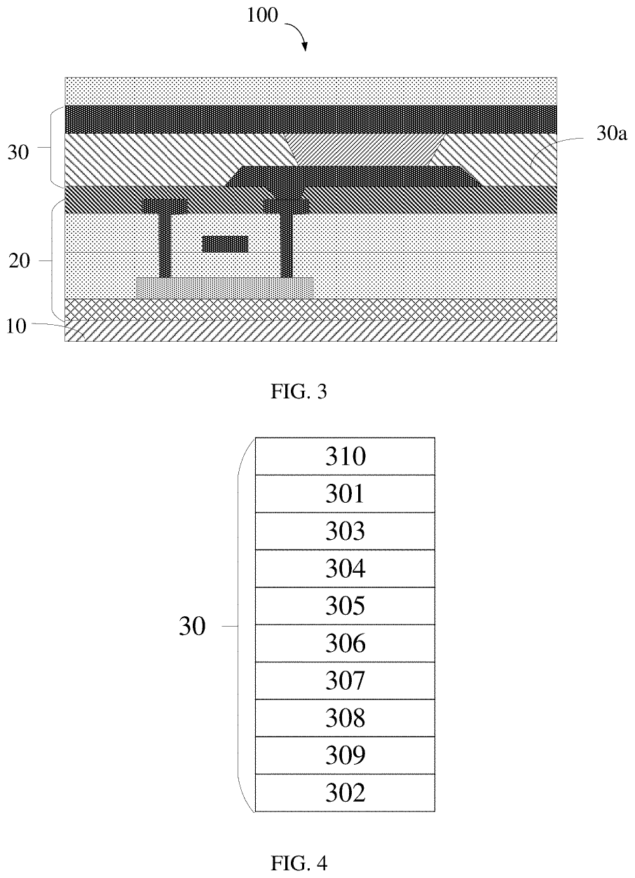

Please refer to FIG. 3 and FIG. 4, an organic light emitting diode (OLED) display panel 100 of one embodiment of the present application includes a substrate 10, a thin film transistor layer 20 disposed on the substrate 10, and a plurality of organic light emitting diode devices 30 disposed on the thin film transistor layer 20.

A glass substrate, a quartz substrate etc., can be applied as the substrate 10. In addition, polyimide, polyethylene terephthalate, polyethersulfone, and other transparent plastic substrates with flexibility can also be applied. In some embodiments of the present application, an opaque plastic substrate and a metal substrate can also be applied.

The thin film transistor layer 20 includes a semi-conductor layer, a gate electrode layer, a source and drain metal layer, and insulating layers disposed between the three of them.

The OLED devices 30 include an anode 301, a cathode 302, and a hole injection layer 303, a hole transport layer 304, an electron blocking layer 305, a light emitting layer 306, a hole blocking layer 307, an electron transport layer 308, and an electron injection layer 309 sequentially stacked between the anode 301 and the cathode 302, and a coupling light emitting layer 310 arranged on a side of the cathode 302 away from the electron injection layer 309. The OLED display panel 100 also includes a pixel definition layer 30a configured to arrange the OLED devices 30. The anode 301 is disposed under the pixel definition layer 30a and the cathode 302 and the coupling light emitting layer 310 are disposed upon the pixel definition layer 30a. The hole injection layer 303, the hole transport layer 304, the electron blocking layer 305, the light emitting layer 306, the hole blocking layer 307, the electron transport layer 308, and the electron injection layer 309 are arranged in a plurality of concaves provided in the pixel definition layer 30a. It can be understood that there are no restrictions in OLED devices 30 in the present application. In other embodiments of the present application, each layer of the OLED devices 30 of the present application can be added or omitted without affecting the technical effect of the application. For example, in other embodiments of the present application, only the anode, the cathode, and the hole injection layer and the light-emitting layer sequentially stacked between the anode and the cathode can be included.

In this embodiment, the OLED device 30 can be a top-emitting type OLED device.

It can be understood that the p-type organic semiconductor material of the present application can also be applied in bottom-emitting type OLED devices.

In this embodiment, the anode 301 is a total reflection anode, which can be configured as an ITO-Ag-ITO stacked structure, and a thickness of the anode 301 is 15 nm.

The cathode 302 is a translucent cathode, which can be configured as a stacked structure of a Mg layer and an Ag layer, and a thickness of the Mg layer is 1 nm, and a thickness of the Ag layer is 10 nm.

The hole injection layer 303 is formed by doping a hole transport material with the p-type organic semiconductor material having the hole injection function of the present application. This layer can also be called hole injection and transport layer. The doping is performed by co-evaporation to make the hole transport materials evenly dispersed in the p-type organic semiconductor materials. A thickness of the hole transport material accounts for 0.1%-10% of a thickness of the hole injection layer. The hole transport material and the p-type organic semiconductor material are doped according to a thickness ratio. The thickness and evaporation rate of each materials are monitored by quartz crystal microbalance (QCM) configured on an evaporating machine. In this embodiment, the thickness of the hole injection layer 303 is 10 nm, and the hole transport material accounts for 3% of the thickness of the hole injection layer 303.

A thickness of the hole transport layer 304 is 117 nm. A thickness of the electron barrier 305 is 5 nm. The light emitting layer 306 is a blue light emitting material layer, which includes a blue light host material and a blue light-emitting material. The total thickness of the light-emitting layer 306 is 20 nm, and the blue light-emitting material accounts for 2% of the thickness of the light-emitting layer 306. A thickness of the hole barrier layer 307 is 5 nm.

The electron transport layer 308 is an n-type doped electron transport material layer, which can include 8-hydroxyquinoline-lithium (LiQ). A total thickness of the electron transport layer 308 is 25 nm, and the thickness ratio of the electron transport material layer to the n-type doped material is 1:1.

A material of the electron injection layer 309 includes lithium fluoride (LiF) with a thickness of 1 nm.

A material of the coupling light-emitting layer 310 is an organic small molecular material with a high refractive index, such as the hole transport materials. A thickness of the coupling optical layer 310 is 65 nm.

All layers except the hole injection layer 303 in the OLED device 30 can be consisted of materials commonly used in the art, detailed descriptions are omitted herein.

In the OLED device 30′ according to another embodiment of the present application, the hole injection layer 303′ is composed of the p-type organic semiconductor material, and the thickness of the hole injection layer 303′ is 1 nm to 8 nm, and the thickness of the hole transport layer 304′ is 122 nm. In addition, the OLED device 30′ is same as the OLED device 30 according to the above-mentioned embodiments. The thickness of the hole injection layer 303 can be for example, 5 nm.

[Photoelectric Performance Tests of the OLED Devices]

An OLED device 30″ using HATCN is prepared. The OLED device using HATCN is the same as the OLED device 30 except that HATCN is used as the material of the hole injection layer.

For the OLED device 30″ using HATCN, the OLED device 30 and the OLED device 30′ (device 30″, device 30, and device 30′ in Table 3 respectively), Keithley source measurement system (Keithley 2400 SourceMeter, Keithley 2000) with a calibrated silicon photodiode is used to measure current-luminance-voltage characteristics of the OLEDs. Electroluminescence spectrums are measured by SPEX CCD3000 spectrometer by JY company from France. All measurements are performed in room temperature atmosphere.

The performance data of each OLED is shown in the Table 3 as below.

TABLE 3

| | | | | Maximum |
|---|---|---|---|---|
| | Driving | Chromatic | Luminescent | Current |
| | Voltage | Coordinates | Peak | Efficiency |
| Device | (V) | (x, y) | (nm) | (cd/A) |
| Device30″ | 3.61 | (0.142, 0.045) | 560 | 5.4 |
| Device 30 | 3.31 | (0.142, 0.045) | 560 | 6.9 |
| Device 30′ | 3.32 | (0.142, 0.046) | 561 | 7.2 |

It can be understood from the table 3 that the driving voltage of the OLED device 30 and 30′ using the p-type organic semiconductor material of the present application is lower than that of the OLED device 30″ using HATCN, and the maximum current efficiency is higher than that of the OLED 30″ using HATCN. In addition, a luminescent peak value of the OLED device using the p-type organic semiconductor material of the present application is about 560 nm, which can be used as green light functional layer doping material. Compared with the prior art, the OLED device of the present application has better photoelectric performance.

The above description provides a detailed introduction to the present application. In the present application, specific examples are applied to explain principle and embodiments of the present application. The description of the above embodiments is only used to help understand the present application.

At the same time, for those skilled in the art, according to the thought of the present application, there will be changes in the specific embodiments and application scope. In conclusion, the content of the specification should not be understood as the limitation of the application.

1. A p-type organic semiconductor material having a molecular structure represented by 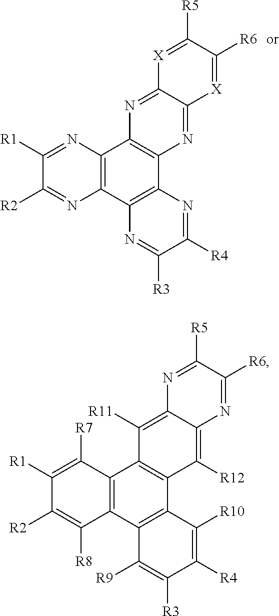
wherein X is a carbon atom or a nitrogen atom, and each of R7, R8, R9, R10, R11, R12 is selected from a first group of substituents;
when R1 and R2 are not bonded to form a ring, each of R1 and R2 is selected from the first group of substituents;
when R1 and R2 are bonded to form the ring, a structure of the ring bonded with R1 and R2 is selected from one of 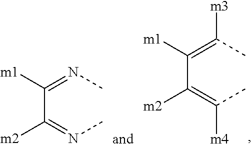
and each of m1, m2, m3, and m4 is selected from a second group of substituents;
when R3 and R4 are not bonded to form a ring, R3 and R4 are selected from the first group of substituents;
when R3 and R4 are bonded to form the ring, a structure of the ring bonded with R3 and R4 is selected from one of 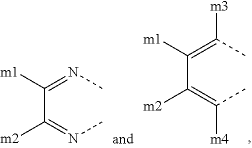
and each of m1, m2, m3, and m4 is selected from the second group of substituents;
when R5 and R6 are not bonded to form a ring, each of R5 and R6 is selected from the first group of substituents;
when R5 and R6 are bonded to form the ring, a structure of the ring bonded with R5 and R6 is selected from one of 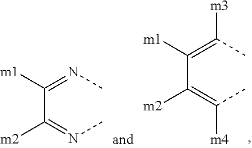
and each of m1, m2, m3, and m4 is selected from the second group of substituents;
wherein the first group of substituents consists of nitro group, nitrile group, cyano group, halogen group, halogen alkyl group, ester group, silyl group, acyl group, sulfonic group, formyl group, carbonyl group, carboxyl group, sulfoxide group, hydroxyl group, alkoxy group, amino group, aromatic amino group, acylamino group, substituted alkene group, substituted phenyl group, substituted heterocyclic group; and
the second group of substituents consists of halogen, CN,

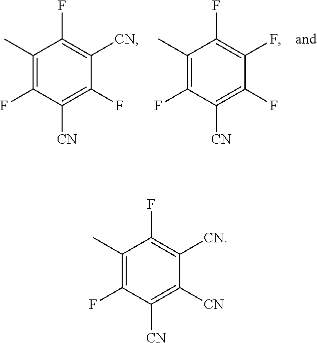

2. The p-type organic semiconductor material of claim 1, wherein the p-type organic semiconductor material is one of following compounds: 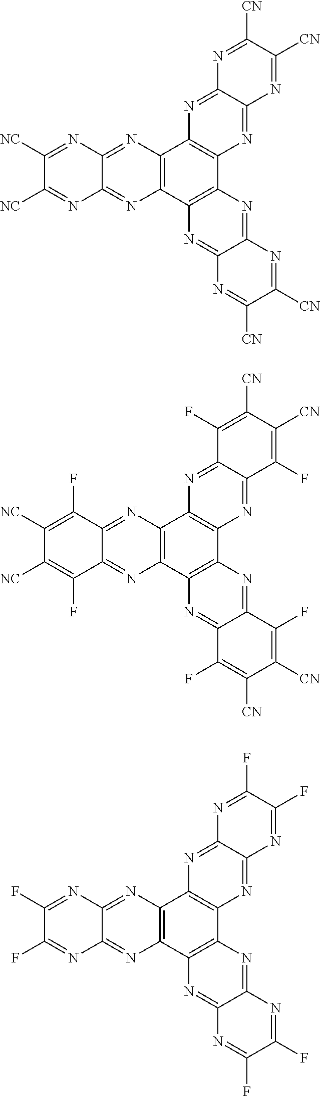 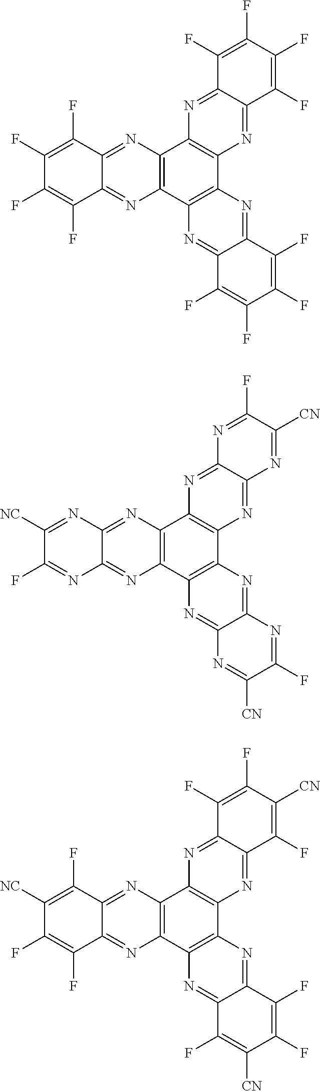 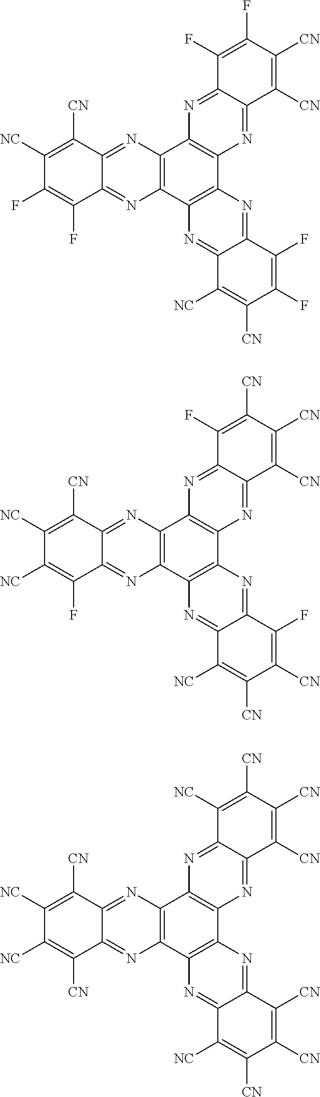 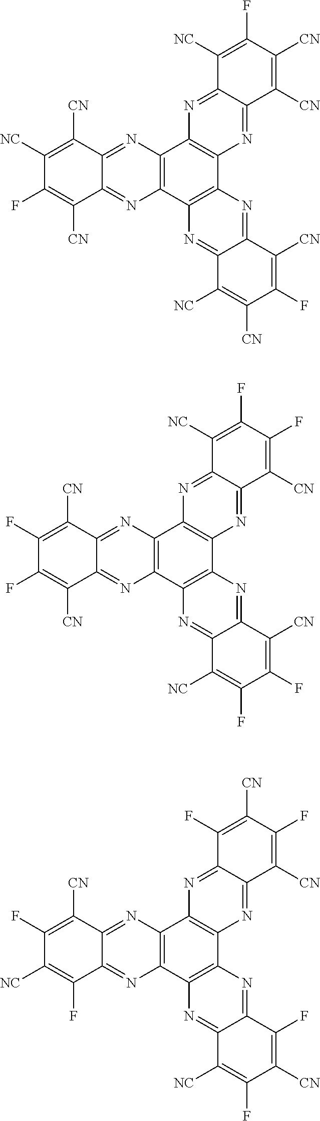 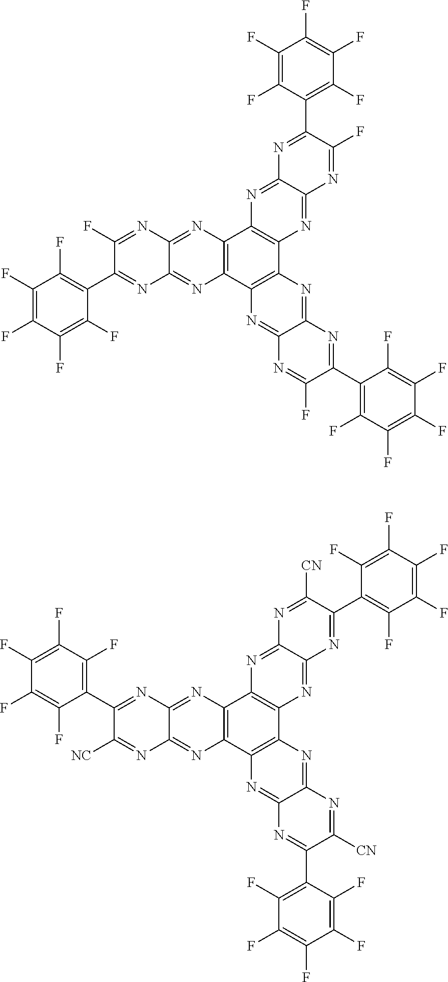 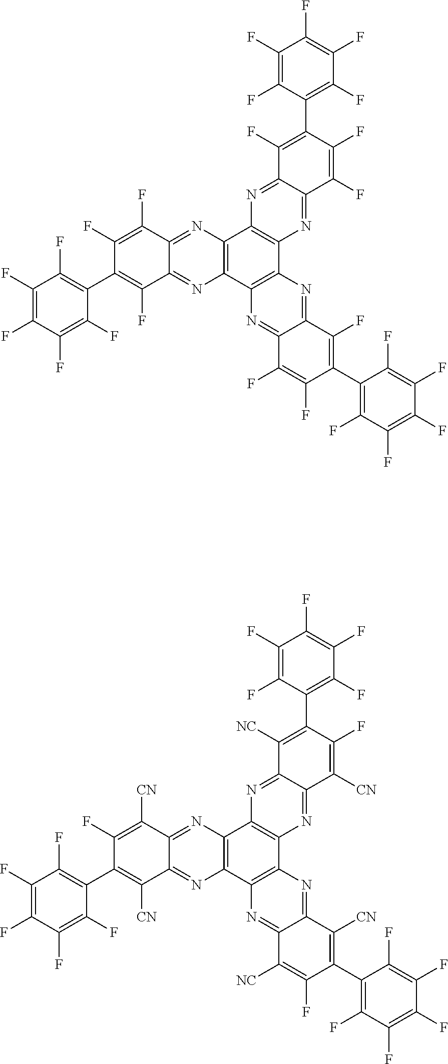 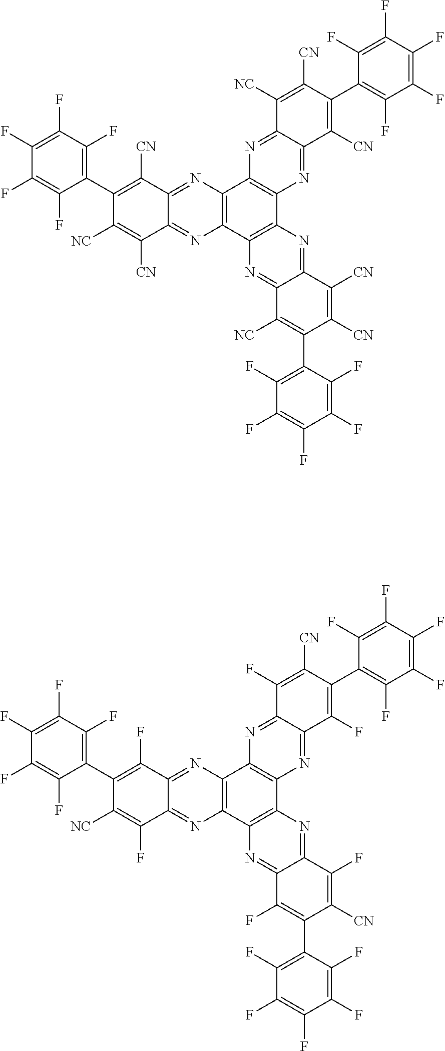 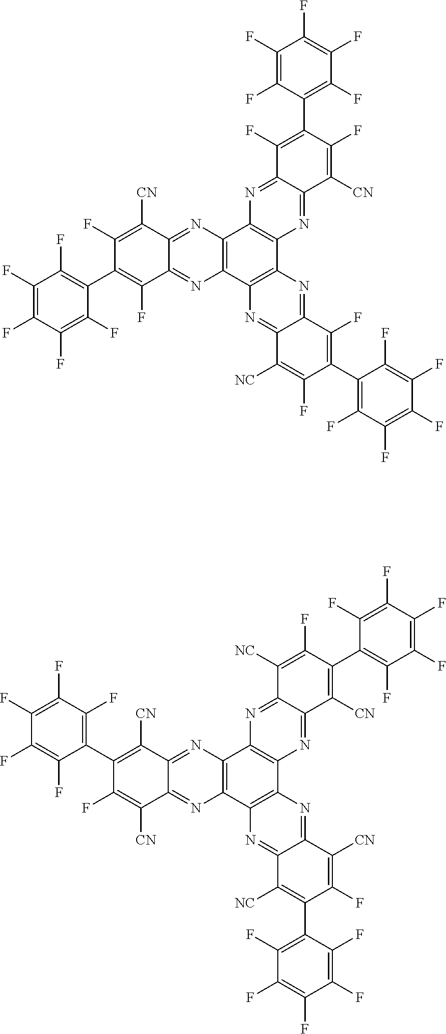 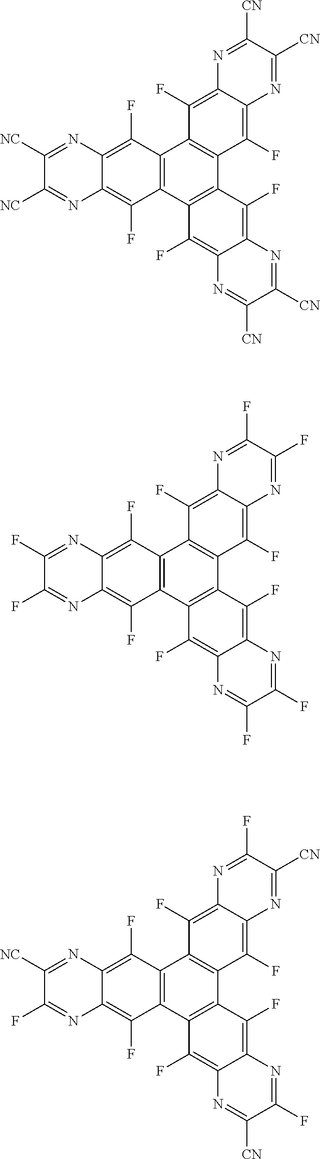 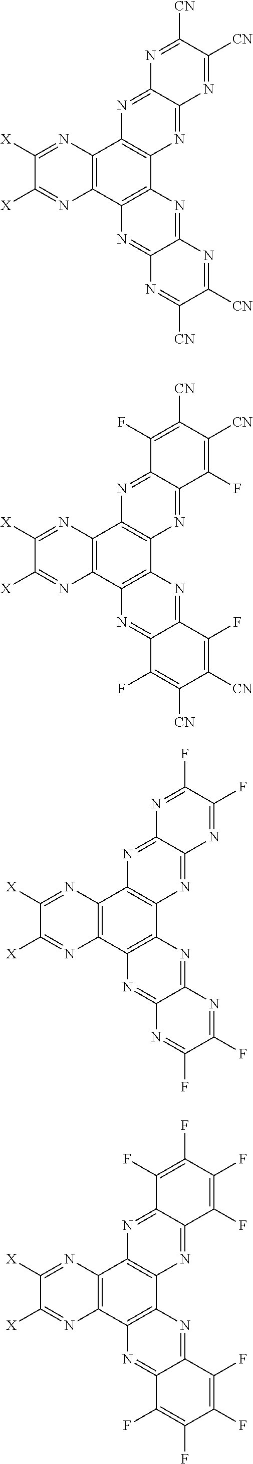 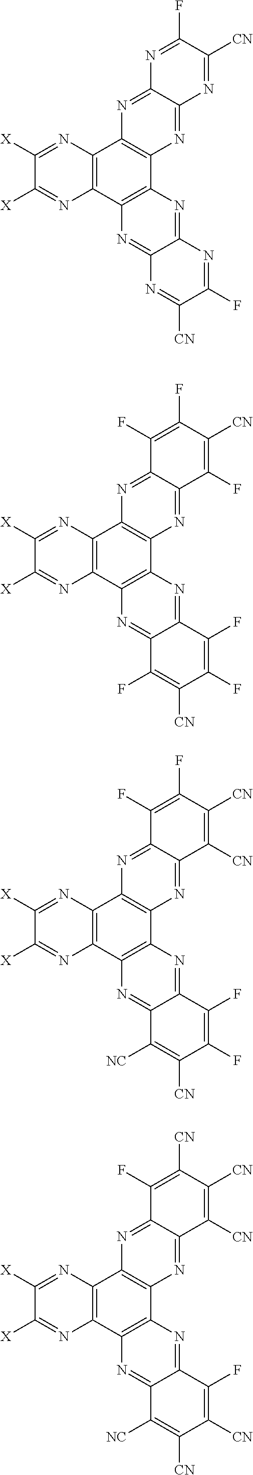 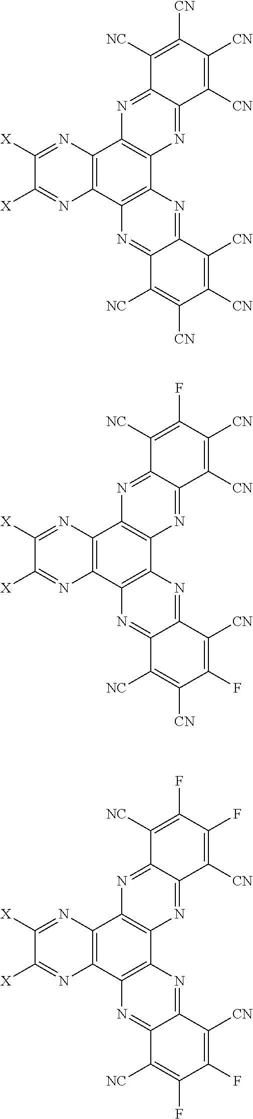 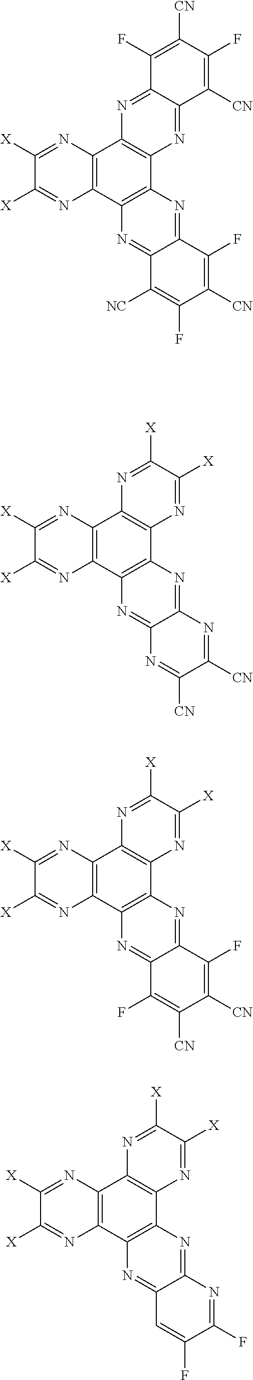 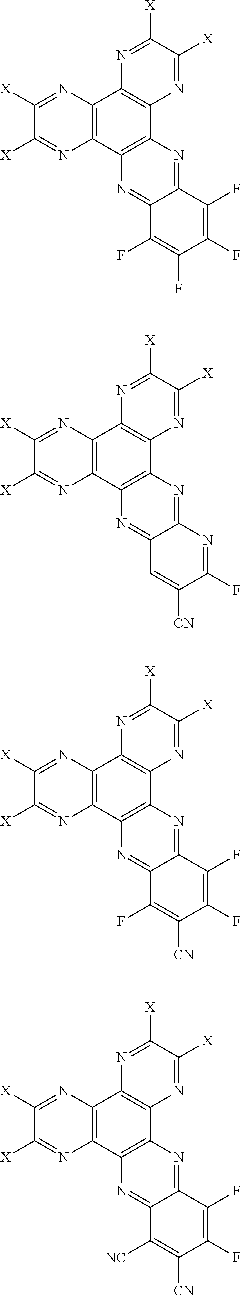 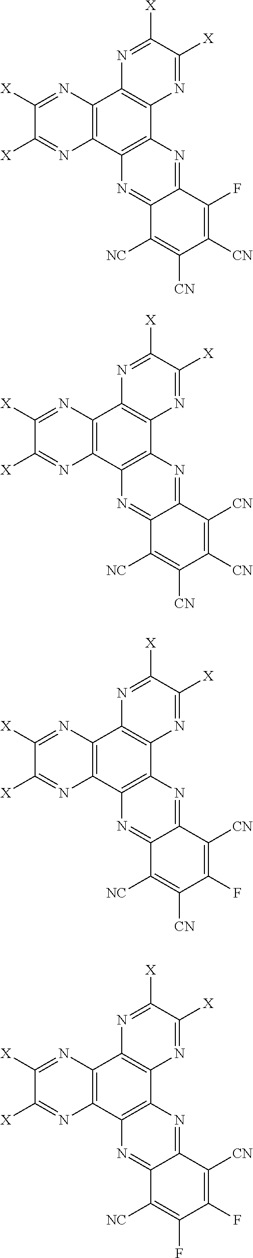 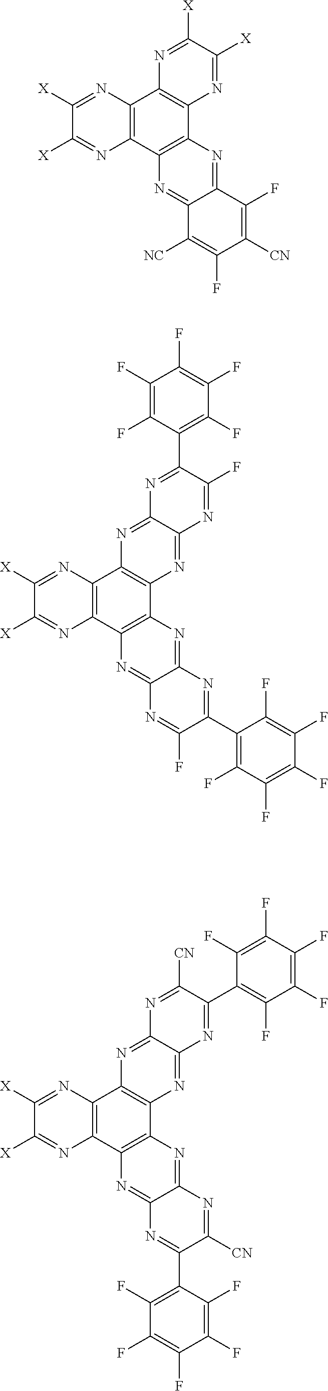 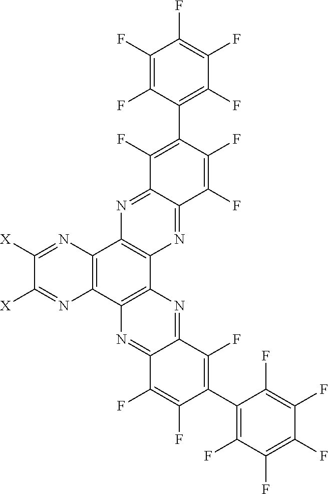 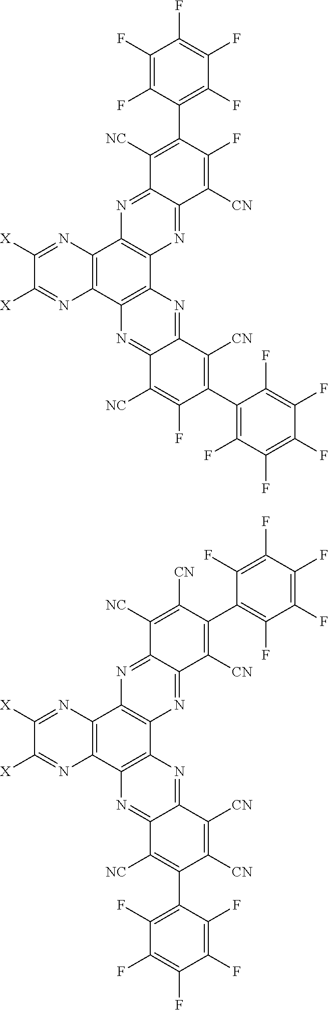 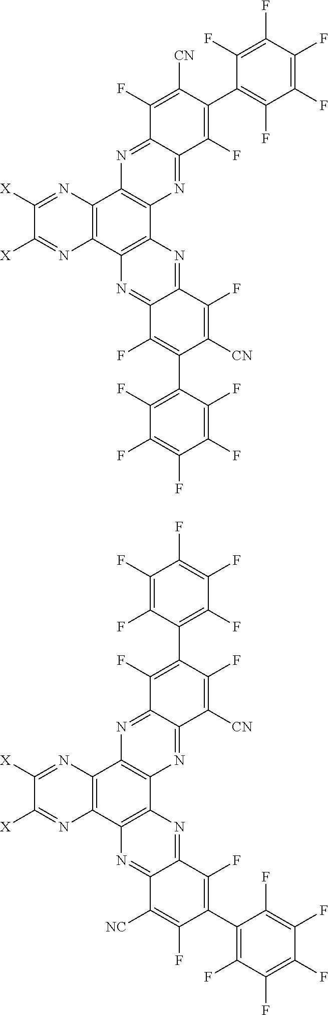 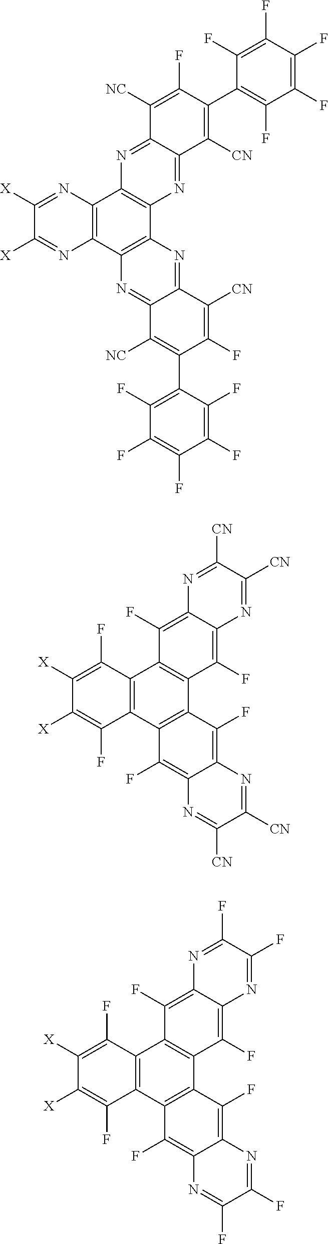 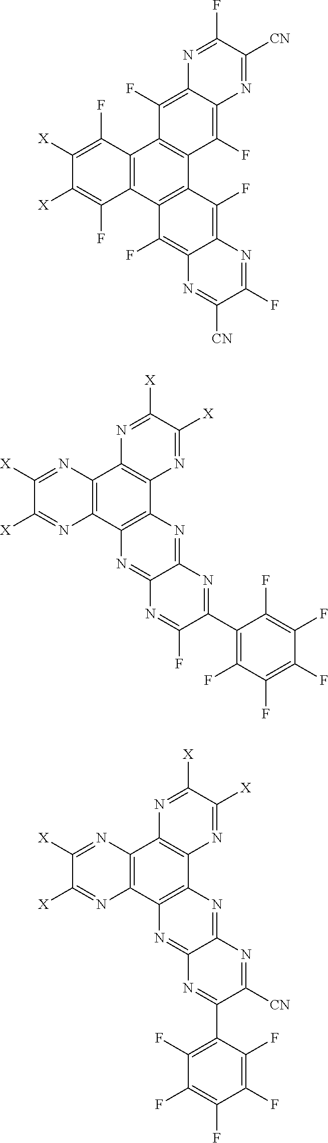 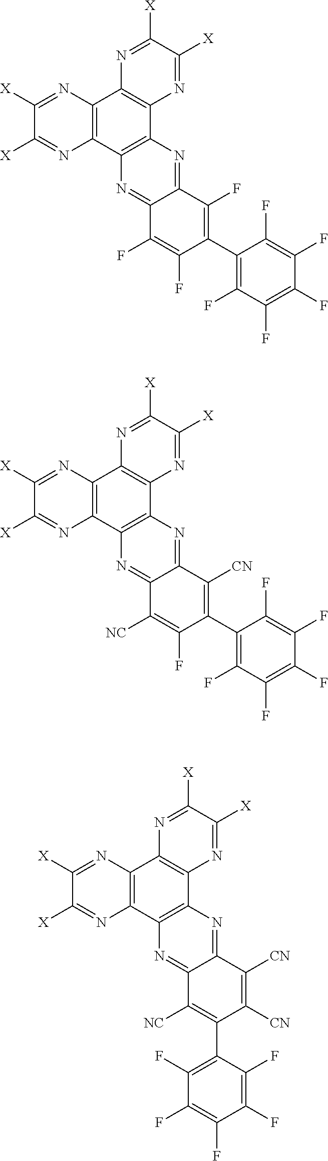 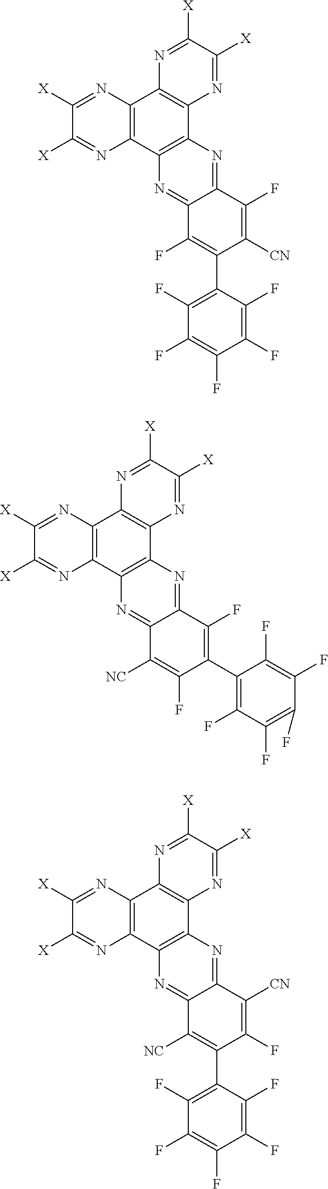 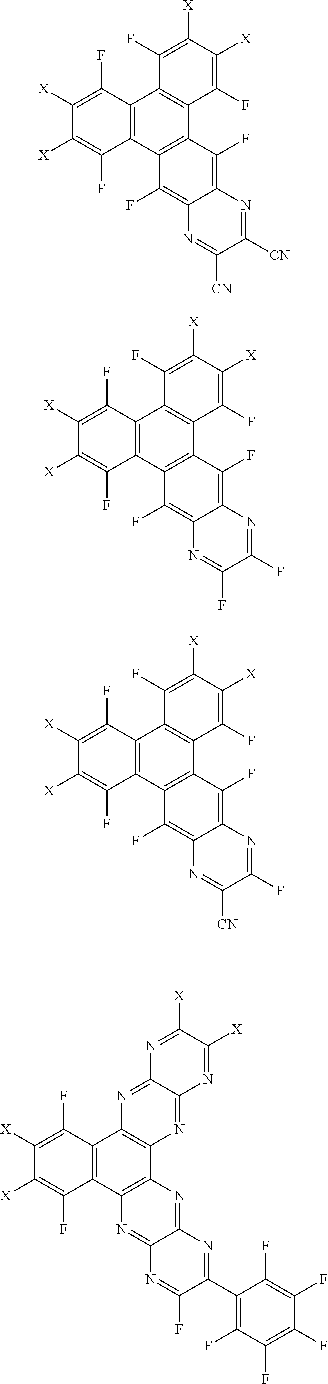 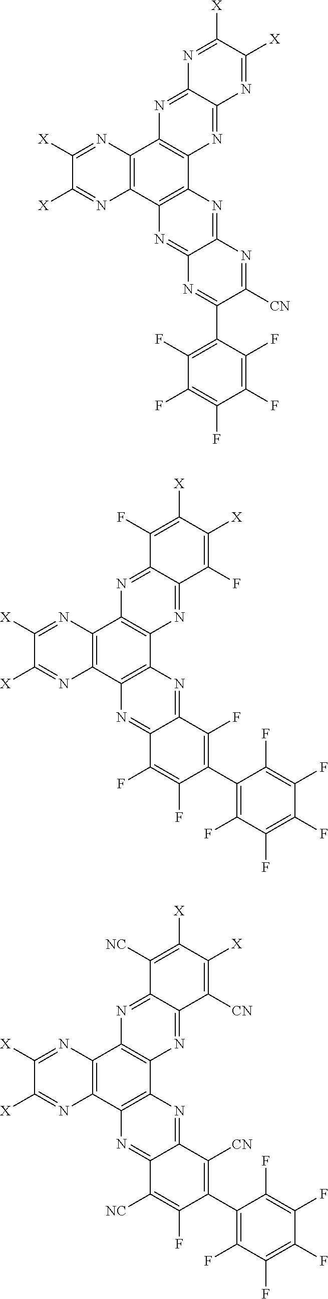 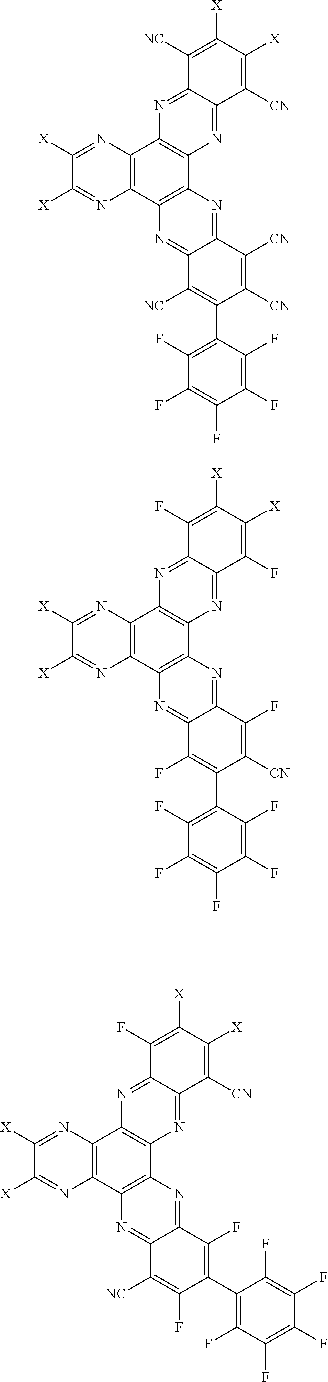 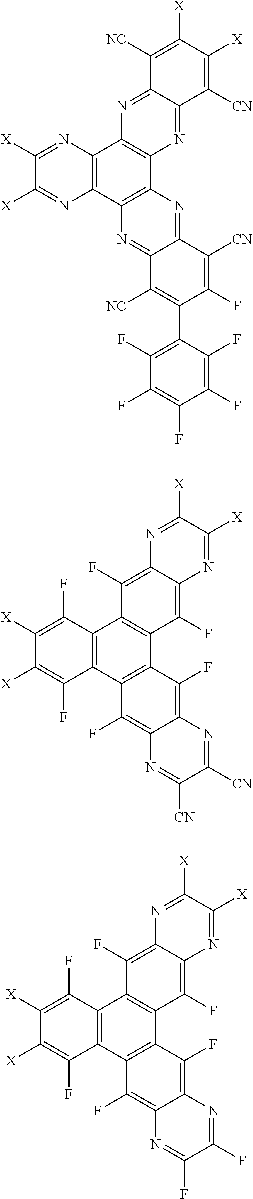 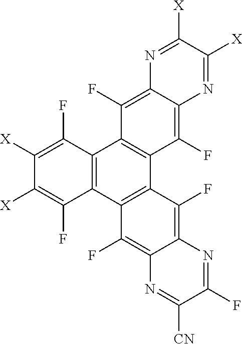
wherein each X is independently selected from one of —CN, —F,

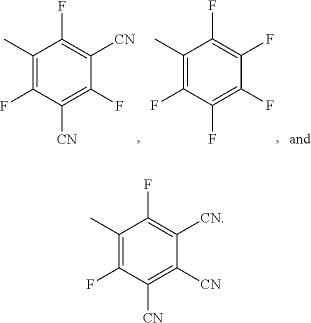

3. A manufacturing method of a p-type organic semiconductor material, comprising following steps:
cyclizing a first reactant containing diketone with a second reactant containing diamine to produce the p-type organic semiconductor material, wherein the p-type organic semiconductor material has a molecular structure represented by 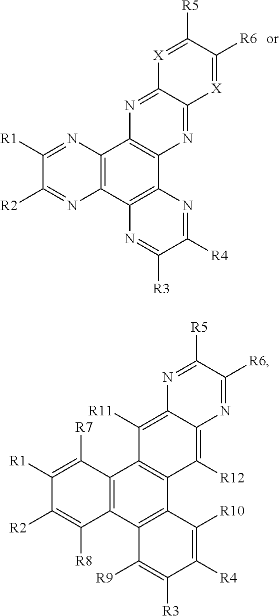
wherein X is a carbon atom or a nitrogen atom, and each of R7, R8, R9, R10, R11, R12 is selected from a first group of substituents;
when R1 and R2 are not bonded to form a ring, each of R1 and R2 is selected from the first group of substituents;
when R1 and R2 are bonded to form the ring, a structure of the ring bonded with R1 and R2 is selected from one of 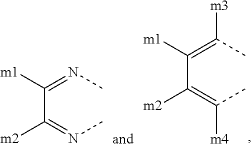
and each of m1, m2, m3, and m4 is selected from a second group of substituents;
when R3 and R4 are not bonded to form a ring, R3 and R4 are selected from the first group of substituents;
when R3 and R4 are bonded to form the ring, a structure of the ring bonded with R3 and R4 is selected from one of 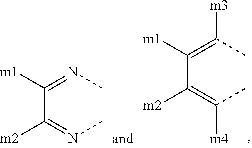
and each of m1, m2, m3, and m4 is selected from the second group of substituents;
when R5 and R6 are not bonded to form the ring, each of R5 and R6 is selected from the first group of substituents;
when R5 and R6 are c bonded to form the ring, a structure of the ring bonded with R5 and R6 is selected from one of 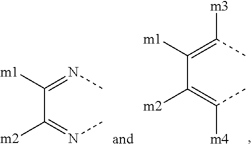
and each of m1, m2, m3, and m4 is selected from the second group of substituents;
wherein the first group of substituents consists of nitro group, nitrile group, cyano group, halogen group, halogen alkyl group, ester group, silyl group, acyl group, sulfonic group, formyl group, carbonyl group, carboxyl group, sulfoxide group, hydroxyl group, alkoxy group, amino group, aromatic amino group, acylamino group, substituted alkene group, substituted phenyl group, substituted heterocyclic group; and
the second group of substituents consists of halogen, CN

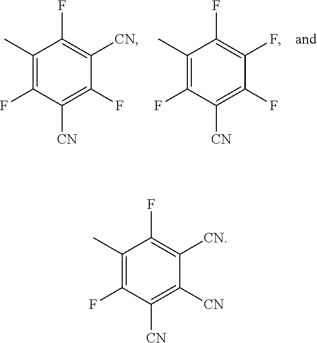

4. The manufacturing method of the p-type organic semiconductor material of claim 3, wherein the first reactant has a molecular structure represented by

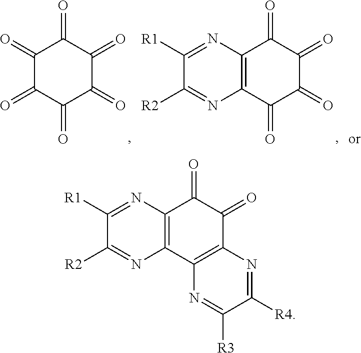

5. The manufacturing method of the p-type organic semiconductor material of claim 4, wherein
when the first reactant is 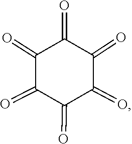 [SMILES: O=C1C(=O)C(=O)C(=O)C(=O)C1=O]
the second reactant comprises 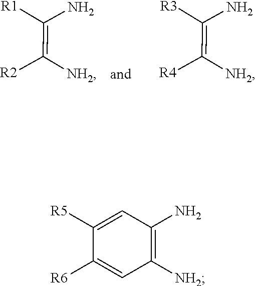
when the first reactant is 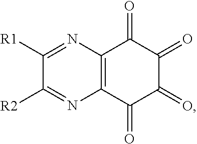
the second reactant comprises 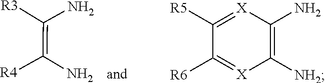
and
when the first reactant is 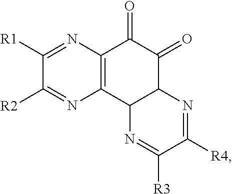
the second reactant comprises

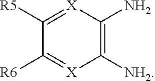

6. The manufacturing method of the p-type organic semiconductor material of claim 5, wherein 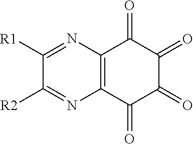
is produced by cyclizing 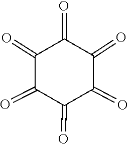 [SMILES: O=C1C(=O)C(=O)C(=O)C(=O)C1=O]
with

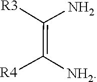

7. The manufacturing method of the p-type organic semiconductor material of claim 5, wherein 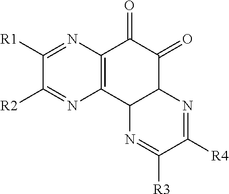
is produced by one or two steps of cyclization reaction of

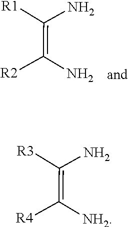

8. The manufacturing method of the p-type organic semiconductor material of claim 3, wherein the first reactant has a molecular structure represented by 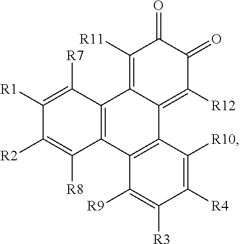
and the second reactant has a molecular structure represented by

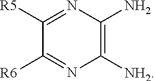

9. The manufacturing method of the p-type organic semiconductor material of claim 8, wherein the manufacturing method further comprises producing 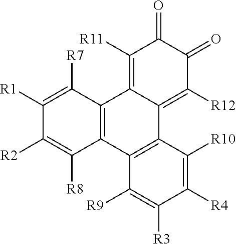
through substitution reaction of

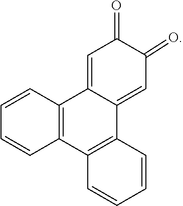 [SMILES: O=C1C=c2c(c3ccccc3c3ccccc23)=CC1=O]

10. The manufacturing method of the p-type organic semiconductor material of claim 3, wherein a molar ratio of the first reactant to the second reactant is 1:1 to 1:10.

11. The manufacturing method of the p-type organic semiconductor material of claim 3, wherein a reaction condition of the step of cyclizing the first reactant containing diketone with the second reactant containing diamine is as below:
acid catalyst is added under protection of nitrogen, and a reaction temperature is 25° C. to 100° C.

12. An organic light-emitting diode display panel, comprising a substrate and a plurality of organic light-emitting diode (OLED) devices disposed on the substrate, wherein each of the OLED devices comprises an anode, a cathode, and a hole injection layer, a hole transport layer, a light-emitting layer, an electron transport layer, and an electron injection layer sequentially stacked between the anode and the cathode, and the hole injection layer comprises the p-type organic semiconductor material of claim 1.

13. The organic light-emitting diode display panel of claim 10, wherein the hole injection layer is composed of the p-type organic semiconductor material, and a thickness of the p-type organic semiconductor material is 1 nm to 8 nm.

14. The organic light-emitting diode display panel of claim 10, wherein the hole injection layer is composed of a hole injection material and the p-type organic semiconductor material, and a thickness of the hole injection material accounts for 0.1% to 10% of a thickness of the hole injection layer.
